(19) United States
(12) Patent Application Publication
(10) Pub. No.: US 2021/0246160 A1
(43) Pub. Date: Aug. 12, 2021

(54) BIOMOLECULE ISOLATION AND INHIBITOR REMOVAL

(71) Applicant: QIAGEN SCIENCES LLC, Germantown, MD (US)

(72) Inventors: Heather CALLAHAN, Escondido, CA (US); Eddie W. ADAMS, San Diego, CA (US)

(21) Appl. No.: 17/049,748

(22) PCT Filed: Apr. 17, 2019
(86) PCT No.: PCT/US2019/027970

§ 371 Date: Oct. 22, 2020

Related U.S. Application Data
(60) Provisional application No. 62/662,066, filed on Apr. 24, 2018.

(51) Int. Cl. C07K 1/16; C07K 1/30

(52) U.S. Cl. CPC C07K 1/16; C12N 15/1003; C07K 1/30

(57) ABSTRACT

The present disclosure provides methods for isolating proteins and optionally nucleic acids from a sample, comprising: (a) contacting a sample, a lysate of the sample, a supernatant of the lysate, or a portion of the sample, the lysate or the supernatant with one or more first agents selected from low molecular weight carboxylates and sulfate and one or more second agents that are multivalent (e.g., trivalent) salt(s) to generate a mixture, (b) separating the mixture of step (a) into a solid phase and a liquid phase, wherein the one or more second agents are primarily in the solid phase, and (c) isolating proteins and optionally nucleic acids from the liquid phase of step (b). Compositions and kits useful in such methods are also disclosed.

### BACKGROUND

### Technical Field

The present disclosure relates to biomolecule (e.g., proteins, DNA and RNA) separation and/or isolation and inhibitor removal from a sample, including a complex sample (e.g., a soil or stool sample).

### Description of the Related Art

Isolating biomolecules (e.g., proteins, DNA and RNA) with high yields and purity is fundamentally important in various fields, including molecular biology, disease diagnosis, forensics, food science, and environmental sciences. Currently, only a few products for isolating all of proteins, DNA and RNA from certain samples are commercially available, none of which are capable of isolating all three biomolecules while effectively depleting inhibitors from complex samples, such as stool samples. The most commonly used method for co-extraction of all three biomolecules without using a commercially available kit uses TRIZOL® Reagent (Ambion) to sequentially extract DNA and RNA followed by protein solubilization. However, this method is both hazardous and time consuming to perform.

### SUMMARY

The present disclosure provides methods, compositions and kits for isolating proteins and optionally nucleic acids while depleting contaminating molecules from a sample.

In one aspect, the present disclosure provides a method for isolating proteins from a sample, comprising:

(a) contacting a sample, a lysate of the sample, a supernatant of the lysate, or a portion of the sample, the lysate or the supernatant with one or more first agents selected from low molecular weight carboxylates, low molecular weight sulfates, carboxylate polymers, sulfonated polymers, or mixtures thereof, and one or more second agents that are multivalent salt(s) to generate a mixture;

(b) separating the mixture of step (a) into a solid phase and a liquid phase, wherein the one or more first agents are primarily in the liquid phase, and wherein the one or more second agents are primarily in the solid phase, and

(c) isolating proteins from the liquid phase of step (b).

In a related aspect, the present disclosure provides a method for sequentially separating and optionally isolating DNA, RNA and proteins from a sample, comprising:

(a) contacting a sample, a lysate of the sample, a supernatant of the lysate, or a portion of the sample, the lysate or the supernatant with one or more first agents selected from low molecular weight carboxylates, low molecular weight sulfates, carboxylate polymers, sulfonated polymers, or mixtures thereof, and one or more second agents that are multivalent salts to generate a mixture;

(b) separating the mixture of step (a) into a solid phase and a liquid phase, wherein the one or more first agents are primarily in the liquid phase, and wherein the one or more second agents are primarily in the solid phase;

(c) separating and optionally isolating DNA from the liquid phase of step (b), comprising:
(1) contacting the liquid phase of step (b) with a first solid support under conditions so that DNA in the liquid phase of step (b) binds to the first solid support,
(2) optionally washing the DNA bound to the first solid support in step (c)(1), and
(3) optionally eluting the DNA optionally washed in step (c)(2) from the first solid support;

(d) separating and optionally isolating RNA from the flow through obtained from step (c)(1), comprising:
(1) contacting the flow through obtained from step (c)(1) with a second solid support under conditions so that RNA in the flow through obtained from step (c)(1) binds to the second solid support,
(2) optionally washing the RNA bound to the second solid support in step (d)(1), and
(3) optionally eluting the RNA optionally washed in step (d)(2) from the second solid support, and

(e) separating and optionally isolating protein from the flow through obtained from step (d)(1), comprising:
(1) contacting the flow through obtained from step (d)(1) with a third solid support under conditions so that proteins in the flow through obtained from step (d)(1) bind to the second solid support,
(2) optionally washing the proteins bound to the third solid support in step (e)(1), and
(3) optionally eluting the protein optionally washed in step (e)(2) from the third solid support.

In another aspect, the present disclosure provides a composition for removing inhibitors during protein isolation from a sample, comprising, consisting essentially of, or consisting of:

(i) one or more first agents selected from low molecular weight carboxylates, low molecular weight sulfates, carboxylate polymers, sulfonated polymers, or mixtures thereof,

(ii) one or more second agents that are multivalent salt(s), and

(iii) optionally water,

wherein the one or more first agents are capable of maintaining water solubility upon coordination of the multivalent cation of the second agent, and

wherein

(A) the one or more first agents are selected from amino acids; salts of short chain fatty acids, such as sodium butyrate; sodium polystyrene sulfonate; sodium polyacrylic acid; preferably ammonium glycolate, sulfoacetic acid, ammonium formate, cesium acetate, beta-alanine, guanidine sulfate, histidine, glycine, and combinations thereof, and the one or more second agents are selected from aluminum sulfate, erbium (III) acetate, erbium (III) chloride, holmium chloride, zirconium (IV) chloride, hafnium (IV) chloride, aluminum ammonium sulfate, aluminum ammonium sulfate dodecahydrate, aluminum potassium sulfate, aluminum chlorohydrate, calcium oxide, iron (III) chloride, iron (II) sulfate, magnesium chloride, aluminum ammonium sulfate, aluminum ammonium sulfate dodecahydrate, aluminum chloride, and combinations thereof,

OR

(B) the one or more first agents are selected from amino acids; salts of short chain fatty acids, such as sodium butyrate; sodium polystyrene sulfonate; sodium polyacrylic acid; preferably ammonium sulfate, ammonium glycolate, sulfoacetic acid, ammonium formate, sodium acetate, cesium acetate, ammonium acetate, beta-alanine, guanidine sulfate, histidine, glycine, and combinations thereof and the one or more second agents are selected from erbium (III) acetate, erbium (III) chloride, holmium chloride, zirconium (IV) chloride, hafnium (IV) chloride, aluminum chloride, and combinations thereof.

In another aspect, the present disclosure provides a kit for isolating proteins from a sample, comprising:

(a) the composition disclosed herein, or

(b) the one or more first agents and the one or more second agents of the composition disclosed herein provided separately.

### BRIEF DESCRIPTION OF THE DRAWINGS

FIG. 1 shows gel electrophoresis of DNA isolated according to Example 1.

FIG. 2 shows gel electrophoresis of DNA (upper panel), RNA (middle panel), and proteins (lower panel) isolated according to Example 2.

FIG. 3 shows gel electrophoresis of DNA isolated according to Example 3.

FIG. 4 shows gel electrophoresis of DNA isolated according to Example 4.

FIG. 5 shows gel electrophoresis of DNA isolated according to Example 5.

FIG. 6 shows gel electrophoresis of DNA (upper panel), RNA (middle panel), and proteins (lower panel) isolated according to Example 6.

### DETAILED DESCRIPTION

The present disclosure provides methods, compositions and kits for isolating biomolecules (e.g., proteins, DNA and RNA) while depleting contaminating molecules from biological or environmental samples, especially from complex samples such as environmental like soil samples and stool samples.

The methods disclosed herein deplete contaminating molecules or inhibitors from a complex sample or lysate in such a fashion that the majority of all soluble DNA, RNA and protein remain in solution during a precipitation step. This affords a contaminant-depleted supernatant to be further processed to isolate protein and optionally DNA and/or RNA.

One significant difference between the methods disclosed herein and related existing techniques for depleting contaminating molecules is that the existing techniques aim at isolating nucleic acids and use ammonium acetate or similar compounds to remove proteinaceous inhibitors from lysates while the methods disclosed herein maintain proteins in solution. Because existing techniques deplete cellular proteins by design, they represent an unsuitable methodology for multi-analyte studies wherein one wishes to study the full genomic, transcriptomic and proteomic contribution to a data set. In contrast, the methods disclosed herein enable to effectively remove inhibitors (e.g., PCR or RT-PCR inhibitors) from complex cellular lysates (e.g., those generated from soil and stool) while maintaining as intact a protein profile as possible.

An additional challenge to preserving accurate protein profiles with the existing techniques comes with the use of aluminum ammonium sulfate dodecahydrate to further deplete PCR-inhibitory compounds from lysates according to existing technologies. While not wishing to be bound by theory, it is believed that aluminum undergoes coordination not only by hard Lewis bases, the carboxylic acids, primary and secondary amines, and phenolic hydroxyl groups found in sample contaminants (e.g., humic acid in soil and stercobilin in stool), but also by those same functional groups present in protein amino acid side chains. Thus, to perform inhibitor depletion via aluminum coordination with its concomitant reduction in coordination complex water solubility, one needs to provide a means to competitively screen protein functional groups from interaction with aluminum.

It is the finding of the present inventors that such a “molecular screen” can be achieved through the inclusion of low molecular weight carboxylates and/or sulfates during the aluminum precipitation step for removing contaminating molecules, such as PCR or RT-PCR inhibitors. By substituting ammonium acetate at protein-precipitating concentrations with compounds (e.g., beta-alanine) capable of forming weak water-soluble coordination complexes with aluminum or with ammonium acetate at lower concentrations, the protein precipitating effects of ammonium acetate are eliminated, and aluminum coordination by protein side chains are blocked or reduced. Using the methods provided herein, excellent retention of nucleic acid and protein yields from complex samples, such as stool microbiome or environmental samples, can be achieved whereas existing techniques lead to significant losses of nucleic acid and proteins.

In addition to being able to integrating inhibitor removal with protein (and optionally DNA and RNA) separation and/or isolation as described above, certain embodiments of the methods disclosed herein have one or both of the following additional advantages:

(1) The methods disclosed herein are able to efficiently separate and/or isolate and purify all three biomolecules (DNA, RNA, protein) in a sequential manner without the need for splitting the starting sample. This not only optimizes yields for each biomolecule but also prevents dilution of rare genes, transcripts and proteins. It also enables a direct comparison of DNA transcription to RNA translation and the final protein products.

(2) The methods disclosed herein may use a spin column format for protein purification. DNA and RNA extraction kits are widely used because of their simplicity in purification by reversible binding to silica matrices. The methods disclosed herein may apply this same technology to protein purification, streamlining protein isolation, making it more user-friendly, enabling automation, and facilitating scale-up and high throughput.

In the following description, any ranges provided herein include all the values in the ranges.

It should also be noted that the term “or” is generally employed in its sense including “and/or” (i.e., to mean either one, both, or any combination thereof of the alternatives) unless the content dictates otherwise.

Also, as used in this specification and the appended claims, the singular forms “a,” “an,” and “the” include plural referents unless the content dictates otherwise.

The terms “include,” “have,” “comprise” and their variants are used synonymously and to be construed as non-limiting.

The term “a combination thereof” as used herein refers to one of the all possible combinations of the listed items preceding the term. For example, “A, B, C, or a combination thereof” is intended to refer to any one of: A, B, C, AB, AC, BC, or ABC. Similarly, the term “combinations thereof” as used herein refers to all possible combinations of the listed items preceding the term. For instance, “A, B, C, and combinations thereof” is intended to refer to all of: A, B, C, AB, AC, BC, and ABC.

### Methods

In one aspect, the present disclosure provides a method for isolating proteins from a sample that comprises:

(a) contacting a sample, a lysate of the sample, a supernatant of the lysate, or a portion of the sample, the lysate or the supernatant with one or more first agents selected from low molecular weight carboxylates, low molecular weight sulfates, carboxylate polymers, sulfonated polymers, or mixtures thereof, and one or more second agents that are multivalent salt(s) to generate a mixture,

(b) separating the mixture of step (a) into a solid phase and a liquid phase, wherein the one or more first agents are primarily in the liquid phase, and wherein the one or more second agents are primarily in the solid phase, and

(c) isolating proteins from the supernatant of step (b).

### Sample Lysis

The method provided herein is useful in isolating proteins (and preferably nucleic acids as well) from any samples that contain such biomolecules, including biological samples, environmental samples and food samples, especially those containing inhibitors that, if present in the preparation of isolated biomolecules, would interfere with downstream analysis of isolated biomolecules.

The term “biological sample” as used herein refers to a sample obtained from or produced by a biological subject, including but are not limited to, organs, tissues, cells, body fluid (e.g., blood, blood plasma, serum, cerebrospinal fluid, or urine), swab samples, stool samples, and plant samples (e.g., seeds, leaves, roots, stems, flowers, cells or tissues from plant tissue culture). A biological sample may be of prokaryotic origin or eukaryotic origin. In some embodiments, the biological sample is mammalian, especially human.

The method provided herein is especially useful in isolating proteins (and preferably nucleic acids as well) from stool samples. Analysis of biomolecules from stool samples allows detection of bacterial and viral infectious agents, monitoring of changes resulting from diet, use of probiotics and antibiotics, and detection of tumor-specific changes, which may be used as a parameter in the early diagnosis of tumors of the digestive tract.

The term “environmental sample” as used herein refers to any environmental material (i.e., a material contained in the earth and space) that contains a biomolecule (e.g., protein, DNA, and RNA). The environmental materials may be materials in soil, water, and air. The biomolecules include those from either live or dead organisms in the environmental materials.

The term “soil” as used herein refers to environmental samples of soil (e.g., potting mixtures, mud), sediment (e.g., marine sediment, lake sediment, river sediment), manure (e.g., poultry, like chicken or turkey, manure, horse manure, cattle manure, goat manure, sheep manure), landfill, compost, and the like.

The term “food sample” as used herein refers to materials, substances or compositions for consumption by animals (e.g., human), including raw food, processed food, meat, fish, poultry, vegetables, eggs, dairy products, bakery products, chocolate, peanut butter, beverages, and the like. A food sample may also include a food enrichment culture produced by contacting a food sample with a culture medium and incubating the mixture under conditions suitable for microorganisms if present in the sample to grow.

After a sample is collected, the sample is typically lyzed to release biomolecules before such molecules are isolated. Sample lysis may be performed at the same time as inhibitor removal, and preferably prior to inhibitor removal.

Sample lysis may be performed by physical disruption, chemical lysis, enzymatic lysis, or a combination thereof. Depending on a given sample type and organisms present in the sample, different sample disruption methods may be used. For example, although human cells and viral capsids are easily lysed by salts or detergents, bacterial spores or oocysts require more aggressive chemical, enzymatic or physical methods.

Physical disruption of sample includes sonication, temperature change, mechanical disruption using a mechanical force, shear force, mechanical vibration, or a vortexer, or a combination of such methods. Mechanical disruption may include the use of bead beating and/or homogenizing methods. The beads useful for mechanical disruptions may be made of or comprise glass, ceramic, metal, mineral, or a combination of two or more of such materials. The size of the beads may range from 0.05 mm to 3 mm. Exemplary beads include 0.7 mm garnet beads, 0.15 mm garnet beads, 0.1 mm glass beads, 0.5 mm glass beads, 0.1 mm ceramic beads, 0.5 mm ceramic beads, 1.4 mm ceramic beads, 0.1 mm yttrium-stabilized zirconium beads, 0.5 mm yttrium-stabilized zirconium beads, or a combination of such beads (e.g., 0.1 mm glass beads and 0.5 mm glass beads in the same amount). In certain preferred embodiments, the beads are high density beads with density (g/cc) at least 6.0, such as yttrium-stabilized zirconium beads, cerium stabilized beads, and stainless steel beads. Bead beating may be performed using a vortex mixer with bead tube adapter or bead beater, such as TissueLyzer II (QIAGEN), AMBION™ Vortex Adapter (Thermo Fisher Scientific, Waltham, Mass.) and the Omini Bead Rupter Homogenizer, OMNI Int'l, Kennesaw, Ga.), and various homogenizers by OPS Diagnostics. The speed and duration of bead beating may vary depending on the type and size of the sample (see e.g., Gibbons et al., Bead Beating: A Primer, OPS Diagnostics, LLC). For example, bead beading may be performed at the maximum speed of a bead beater for 1 to 20 minutes, such as 5 to 10 minutes, 10 to 20 minutes, or 5 to 15 minutes.

Enzymatic lysis includes the use of an amylase, cellulase, lipase or the like. However, because such added enzymes may be present in the protein preparation isolated from a sample, preferably, sample disruption other than enzymatic lysis is performed in the methods provided herein.

Chemical lysis includes the use of lytic reagents comprising chaotropic agents. A chaotropic agent disrupts the structure of, and denatures macromolecules such as proteins and nucleic acids. Chaotropic solutes increase the entropy of the system by interfering with intramolecular interactions mediated by non-covalent forces such as hydrogen bounds, van der Waals forces, and hydrophobic effects, on which macromolecular structure and function depend. Exemplary chaotropic agents include guanidinium chloride, guanidine thiocyanate, urea, or lithium salts.

In certain embodiments, the chaotropic agents denature proteins less than the stronger chaotropic agent, guanidinium thiocyanate (GuSCN) or guandinium chloride (GuCl) but more than the weaker chaotropic agent, sodium chloride. Such relatively mild (also referred to as “less aggressive”) chaotropic agents include certain Hofmeister series chaotrope cation/anion combinations wherein a relatively strong anion is combined with a relatively weak cation, or a relatively strong cation is combined with a relatively weak anion.

The Hofmeister series is a classification of ions in order of their ability to salt out or salt in proteins. This series of salts have consistent effects on the solubility of proteins and on the stability of their secondary and tertiary structure. Anions appear to have a larger effect than cations, and exemplary anions are usually ordered as follows:

C₃²⁻>F⁻or SO₄²⁻>HPO₄²⁻>acetate>Cl⁻>Br⁻>NO₃⁻>ClO₃⁻>I⁻>CO₄⁻>SCN⁻

The order of exemplary cations is usually given as follows:

N(CH₃)₄⁺>Cs⁺>Rb⁺>NH₄⁺>K⁺>Na⁺>Li⁺>Mg²⁺>Ca²⁺>guanidinium

Exemplary relatively mild chaotropic agents include NaSCN, NaCO₃, KSCN, NH₄SCN, LiSCN, LiClO₄, guanidine sulfate, and combinations thereof. Preferably, the relatively mild chaotropic agent is NaSCN or NaCO₃.

The relatively mild chaotropic agents may include salts having the strong anion, SCN⁻, paired with a cation weaker than Mg²⁺ in solubilizing proteins; salts having the strong anion, ClO₄⁻, paired with a cation weaker than Mg²⁺ in solubilizing proteins; and salts having the weak anion, C₃²⁻, paired with a cation stronger than NH₄⁺ in solubilizing proteins.

The relatively mild chaotropic agents (e.g., NaSCN) strike a desirable balance between a stronger chaotropic agent such as GuSCN or GuCl and a weaker chaotropic agent such as RbSCN. Such a less aggressive chaotropic agent typically requires an additional mechanism, such as mechanical disruption to lyze a sample, especially a complex sample (e.g., a stool sample). However, the less aggressive chaotropic agent can effectively solubilize nucleic acids and proteins during for example homogenization to make them available for downstream isolation steps. Strong chaotropic agents and detergents (e.g., SDS), on the other hand, can achieve complete cell lysis but at the expense of a significant loss of one or more biomolecules, such as degraded nucleic acids. The less aggressive chaotropic agents are unique in their capacity to balance solubilization of cellular components while minimizing biomolecular degradation.

The concentration of a chaotropic agent in a lytic reagent may be in the range of 0.05 to 5M, such as 0.05 to 0.1M, 0.1 to 0.5M, 0.5 to 1M, 1 to 1.5M, 1.5 to 2M, 2 to 5 M, 0.1 to 1M, 0.1 to 1.5M, 0.1 to 2M, 0.1 to 5M, 0.5 to 1.5M, 0.5 to 2M, 0.5 to 5M, 1 to 2M, or 1 to 5M, preferably 0.05 to 0.5M or 0.5 to 2M. The final concentration of a chaotropic agent in a lysate (i.e., the mixture of a sample and the lytic reagent) may be 0.01 to 4M, such as 0.01 to 0.05M, 0.05 to 0.1M, 0.1 to 0.5M, 0.5 to 1M, 1 to 1.5M, 1.5 to 2M, 2 to 4M, 0.01 to 0.1M, 0.01 to 0.5M, 0.01 to 1M, 0.01 to 1.5M, 0.01 to 2M, 0.01 to 4M, 0.05 to 0.5M, 0.05 to 1M, 0.05 to 1.5M, 0.05 to 2M, 0.05 to 2M, 0.05 to 4M, 0.1 to 1M, 0.1 to 1.5M, 0.1 to 2M, 0.1 to 4M, 0.5 to 1.5M, 0.5 to 2M, 0.5 to 4M, 1 to 2M, or 1 to 4M, preferably 0.05 to 0.5M or 0.5 to 2M.

For example, the concentration of NaSCN in a lytic reagent may be 0.5 to 2M, preferably 0.8 to 1.2M. The final concentration of NaSCN in a lysate (i.e., the mixture of a sample and the lytic reagent) may be 0.1 to 1.8M, preferably 0.5 to 1.1M.

The concentration of Na₂CO₃ in a lytic reagent may be 0.05 to 0.2M, preferably 0.08 to 0.12M. The final concentration of Na₂CO₃ in a lysate (i.e., the mixture of a sample and the lytic reagent) may be 0.01 to 0.4M, preferably 0.04 to 0.15 M.

If multiple chaotropic agents, in particular multiple relatively mild chaotropic agents are present in a lytic reagent, the total concentration of the chaotropic agents in combination in the lytic reagent may be in the range of 0.05 to 5M, such as 0.05 to 0.1M, 0.1 to 0.5M, 0.5 to 1M, 1 to 1.5M, 1.5 to 2M, 2 to 5 M, 0.1 to 1M, 0.1 to 1.5M, 0.1 to 2M, 0.1 to 5M, 0.5 to 1.5M, 0.5 to 2M, 0.5 to 5M, 1 to 2M, or 1 to 5M. The concentration of an individual chaotropic agent in the lytic reagent may be in the range of 0.01 to 4.5M, such as 0.01 to 0.05M, 0.05 to 0.1M, 0.1 to 0.5M, 0.5 to 1M, 1 to 1.5M, 1.5 to 2M, 2 to 4.5 M, 0.01 to 0.1M, 0.01 to 0.5M, 0.01 to 1M, 0.01 to 1.5M, 0.01 to 2M, 0.1 to 1M, 0.1 to 1.5M, 0.1 to 2M, 0.1 to 4.5M, 0.5 to 1.5M, 0.5 to 2M, 0.5 to 4.5M, 1 to 2M, or 1 to 4.5M, preferably 0.01 to 0.5M or 0.1 to 2M. The total final concentration of chaotropic agents in combination in a lysate (i.e., the mixture of a sample and the lytic reagent) may be 0.01 to 4M, such as 0.01 to 0.05M, 0.05 to 0.1M, 0.1 to 0.5M, 0.5 to 1M, 1 to 1.5M, 1.5 to 2M, 2 to 4M, 0.01 to 0.1M, 0.01 to 0.5M, 0.01 to 1M, 0.01 to 1.5M, 0.01 to 2M, 0.01 to 4M, 0.05 to 0.5M, 0.05 to 1M, 0.05 to 1.5M, 0.05 to 2M, 0.05 to 2M, 0.05 to 4M, 0.1 to 1M, 0.1 to 1.5M, 0.1 to 2M, 0.1 to 4M, 0.5 to 1.5M, 0.5 to 2M, 0.5 to 4M, 1 to 2M, or 1 to 4M, preferably 0.05 to 0.5M or 0.5 to 2M. The final concentration of an individual chaotropic agent in the lysate may be 0.001 to 3.5M, such as 0.001 to 0.01M, 0.01 to 0.05M, 0.05 to 0.1M, 0.1 to 0.5M, 0.5 to 1M, 1 to 1.5M, 1.5 to 2M, 2 to 3.5M, 0.001 to 0.1M, 0.001 to 0.5M, 0.001 to 1M, 0.001 to 1.5M, 0.001 to 2M, 0.001 to 3.5M, 0.01 to 0.1M, 0.01 to 0.5M, 0.01 to 1M, 0.01 to 1.5M, 0.01 to 2M, 0.01 to 3.5M, 0.05 to 0.5M, 0.05 to 1M, 0.05 to 1.5M, 0.05 to 2M, 0.05 to 2M, 0.05 to 3.5M, 0.1 to 1M, 0.1 to 1.5M, 0.1 to 2M, 0.1 to 3.5M, 0.5 to 1.5M, 0.5 to 2M, 0.5 to 3.5M, 1 to 2M, or 1 to 3.5M, preferably 0.01 to 0.5M or 0.1 to 2M.

A lytic reagent may further comprise one or more phosphates. Phosphate is especially useful in achieving uniform disruption of soil particles, solubilzing soil organic matter, and extracting humic substances from soil. In addition, without wishing to be bound by theory, it is believed that the free phosphate group (PO₄³⁻) also prevents or reduces complex formation between an inhibitor removing agent (e.g., AlCl₃) and the phosphodiester groups of nucleic acids by competitively interacting with the inhibitor removing agent. Exemplary phosphates include phosphate monobasics, phosphate dibasics, and phosphate tribasics, and other compounds that contain one or more free phosphate groups, such as sodium phosphate monobasic, sodium phosphate dibasic, sodium phosphate, potassium phosphate monobasic, potassium phosphate dibasic, potassium phosphate, ammonium phosphate monobasic, ammonium phosphate dibasic, ammonium phosphate, lithium phosphate monobasic, lithium phosphate dibasic, lithium phosphate, trisodium phosphate, sodium poly(vinylphosphonate), sodium hexametaphosphate, pyrophosphate, sodium triphosphate, sodium polyphosphate, other phosphorus-containing oxyanions, and combinations thereof. The cationic moieties in the phosphates include but are not limited to ammonium, sodium, potassium, and lithium.

The concentration of phosphate in a lytic reagent may be 0.05 to 0.5M, preferably 0.1 to 0.2M. The final concentration of phosphate in a lysate (i.e., the mixture of a sample and the lytic reagent) may be 0.01 to 0.4M, preferably 0.1 to 0.2M.

If multiple phosphates are present in a lytic reagent, the total concentration of phosphates in combination in the lytic reagent may be in the range of 0.05 to 0.5M, preferably 0.1 to 0.2M. The concentration of an individual phosphate in the lytic reagent may be in the range of 0.01 to 0.45M, such as 0.01 to 0.1M, 0.1 to 0.2M, 0.2 to 0.3M, 0.3 to 0.45M, preferably 0.01 to 0.2M. The total final concentration of phosphates in combination in a lysate (i.e., the mixture of a sample and the lytic reagent) may be 0.01 to 0.4M, 0.01 to 0.05M, 0.05 to 0.1M, 0.1 to 0.4M, preferably 0.1 to 0.2M. The final concentration of an individual phosphate in the lysate may be in the range of 0.001 to 0.35M, such as 0.001 to 0.01M, 0.01 to 0.05M, 0.05 to 0.1M, 0.1 to 0.35M, 0.1 to 0.2M, 0.2 to 0.35M, preferably 0.01 to 0.2M.

A lytic reagent may also include one or more detergents, including nonionic, cationic, anionic (sodium dodecyl sulfate) or zwitterionic detergents. Exemplary detergents include sodium dodecyl sulfate (SDS), sarkosy sodium lauryl sarcosinate, cetyltrimethyl ammonium bromide (CTAB), cholic acid, deoxycholic acid, benzamidotaurocholate (BATC), octyl phenol polyethoxylate, polyoxyethylene sorbitan monolaurate, tert-octylphenoxy poly(oxyethylene)ethanol, 1,4-piperazinebis-(ethanesulfonic acid), N-(2-acetamido)-2-aminoethanesulfonic acid, polyethylene glycoltert-octylphenyl ether (TRITON®-100), (1,1,3,3-tetramethylbutyl)phenyl-polyethylene glycol (TRITON® X-114), and combinations thereof.

The total concentration of detergents in combination in a lytic reagent may be in the range of 0.01% to 15% (v/v) if the detergent(s) is liquid or 0.01% to 15% (w/v) if the detergent(s) is solid. The concentration of an individual detergent in the lytic reagent may be in the range of 0.001 to 15%, such as 0.005 to 12%, 0.01 to 10%, 0.1 to 8%, 0.05 to 6%, 0.1 to 4%, 0.5 to 2%, 0.8 to 1%, preferably 0.01 to 15%. The total final concentration of the detergents in combination in a lysate (i.e., the mixture of a sample and the lytic reagent) may be 0.005% to 12%, such as 0.005% to 0.05%, 0.05% to 0.5%, 0.5% to 5%, 5% to 12%, 0.05% to 10%, 0.1% to 10%, or 0.5% to 5%. The total final concentration of an individual detergent in the lytic reagent may be in the range of 0.001 to 12%, such as 0.005 to 10%, 0.01 to 8%, 0.05 to 6%, 0.05 to 6%, 0.1 to 4%, 0.2 to 2%, 0.5 to 1%, preferably 0.001 to 12%.

In certain other embodiments, a lytic reagent does not include any detergent, such as SDS.

A lytic reagent may additionally contain one or more blocking agents that block or reduce the interaction between contaminants in a sample and biomolecules liberated during lysis and solubilization. Exemplary blocking agents include casein, polyacrylic acid and polystyrene sulfonate. Such blocking agents are useful in blocking electrostatic interactions between particles in a sample (e.g., soil particles) having positively charged groups (e.g., metal ions) and DNA, RNA and proteins released from the sample. Such interactions, if not disrupted, can lead to significant decreases in biomolecule yields from the sample.

The total concentration of blocking agents in combination in a lytic reagent may be in the range of 0.01 to 0.5 M of relevant functional group (e.g., carboxylates in the case of polyacrylic acid; sulfonates). The concentration of an individual blocking agent in the lytic reagent may be in the range of 0.001 to 0.5M. The total final concentration of the blocking agents in combination in a lysate (i.e., the mixture of a sample and the lytic reagent) may be in the range of 2 to 400 mM. The final concentration of an individual blocking agent in the lytic reagent may be in the range of 0.2 to 400 mM.

In certain other embodiments, a lytic reagent does not include any blocking agent.

A lytic reagent may further contain one or more salts other than the chaotropic agents or phosphates described above. Exemplary salts include NaCl, NaF, LiCl, NaBr, NaI, RbCl, CsCl, RbBr, CsBr, RbI, CsI, and combinations thereof. The total concentration of the salts in the lytic reagent in combination may be in the range of 10 to 500 mM, such as 30 to 300 mM or 50 to 200 mM. The concentration of an individual salt in the lytic reagent may be in the range of 1 to 500 mM, such as 10 to 200 mM or 25 to 100 mM. In certain other embodiments, a lytic reagent does not include any of such salts (e.g., NaCl).

A lytic reagent may further contain one or more buffer substances so that lysis occurs at a stable pH. The pH of the lytic reagent may be in the range of pH 6 to pH 12, such as pH 6 to pH8, pH 7 to pH9, pH 8 to pH 10, and pH 8 to pH 11, and pH 7 to pH10.

The lysis is preferably performed at a low temperature (e.g., 4° C.) to avoid or reduce protein denaturation. Preferably, proteinase inhibitors (e.g., Halt protease inhibitors from Thermo Fisher) are added to the sample material, the lytic reagent, or a mixture of the sample material and the lytic reagent shortly prior to sample lysis to prevent or reduce protein degradation during sample lysis and subsequent protein isolation. Similarly, a reducing agent (e.g., beta-mercaptoethanol) may be added to the sample material, the lytic reagent, or a mixture of the sample material and the lytic reagent shortly prior to sample lysis to avoid the loss of activity of proteins or enzymes caused by oxidization.

In certain preferred embodiments, the lytic reagent in its solid state comprises, consists essentially of, or consists of a relatively mild chaotropic agent and a phosphate, both as described above. Preferably, the lytic reagent is a solution that comprises, consists essentially of, or consists of an above-described relatively mild chaotropic agent, an above-described phosphate, and water. Preferably, the relatively mild chaotropic agent comprises or is NaSCN or NaCO₃, especially NaSCN. The phosphate preferably comprises or is sodium phosphate dibasic. An exemplary preferred lytic reagent comprises, consists essentially of, or consists of 0.5 to 2M NaSCN and 0.1 to 0.2M Na₂HPO₄. Another exemplary preferred lytic reagent comprises, consists essentially of, or consists of 0.05 to 0.5M Na₂CO₃ and 0.1 to 0.2M Na₂HPO₄.

The lytic reagents that comprise, consist essentially of, or consist of a relatively mild chaotropic agent and a phosphate are preferably used in combination of mechanical disruption (e.g., bead beating) in isolating biomolecules from a complex sample, such as a stool sample. The biomolecules may be of a microbial origin.

The lysate of a sample may be directly used in step (a) in the method disclosed herein. Preferably, the lysate is separated into a liquid phase that comprises biomolecules released from the sample and a solid phase that contains solid particles or residues from the sample by filtration, sedimentation or preferably centrifugation. The resulting liquid phase (i.e., supernatant) or a portion thereof may be used to isolate biomolecules and remove inhibitors.

### Inhibitor Removal

The method provided herein isolates biomolecules (proteins and preferably also nucleic acids) from a sample and removes inhibitors from the isolated biomolecules, allowing effective downstream analysis of such biomolecules. Specifically, step (a) of the method disclosed herein is to contact a sample, a lysate of the sample, a supernatant of the sample, or a portion of the sample, the lysate or the supernatant with one or more first agents selected from low molecular weight carboxylates, low molecular weight sulfates, carboxylate polymers, sulfonated polymers, or mixtures thereof, and one or more second agents that are multivalent salt(s) to generate a mixture. Step (b) is to separate the mixture of step (a) into a solid phase and a liquid phase, wherein the one or more first agents are primarily in the liquid phase, while the one or more second agents are primarily in the solid phase and thus removed from the liquid phase, which is subsequently used in isolating biomolecules.

The first agent useful in the method provided herein is selected from low molecular weight carboxylates, low molecular weight sulfates, carboxylate polymers, sulfonated polymers, or mixtures thereof, and is also referred to as a “molecular screen.” Such an agent is capable of competing with functional groups of proteins in a sample for a limited pool of exogenous multivalent cation (e.g., Al³⁺) and thus screening (i.e., preventing) protein side chains from interacting with the multivalent cation of a multivalent salt. Such screening reduces the amount of proteins that are precipitated by the multivalent salt along with inhibitors and other contaminating substances.

The term “low molecular weight” refers to a molecular weight no more than 500 g/mole, such as no more than 400, 300, 200 or 150 g/mole. Exemplary low molecular weight carboxylates and sulfates include but are not limited to ammonium acetate, ammonium sulfate, ammonium glycolate, sulfoacetic acid, ammonium formate, beta-alanine, guanidine sulfate, histidine, glycine, sodium acetate, cesium acetate, other amino acids (e.g., arginine, asparagine, aspartic acid, cysteine, glutamine, glutamic acid, isoleucine, leucine, lysine, methionine, phenylalanine, proline, serine, threonine, tryptophan, tyrosine, valine), salts of short chain fatty acids (salts of fatty acids containing 3 to 5 carbons, such as sodium butyrate, sodium propionate, sodium isobutyrate, sodium valerate, sodium isovalerate, ammonium butyrate, ammonium propionate, ammonium isobutyrate, ammonium valerate, ammonium isovalerate, cesium butyrate, cesium propionate, cesium isobutyrate, cesium valerate, or cesium isovalerate), and combinations thereof.

The first agents may also include carboxylate polymers, sulfonated polymers, and combinations thereof. Exemplary carboxylate polymers include sodium polyacrylic acid. Exemplary sulfonate polymers include sodium polystyrene sulfonate. The molecular weight of such polymers may be in the range of 5 to 1000 KD, such as 5 to 10 KD, 10 to 100 KD, 100 to 500 KD, 500 to 1000 KD, 5 to 100 KD, 5 to 500 KD, 10 to 500 KD, 10 to 1000 KD, or 100 to 1000 KD.

In certain embodiments, the one or more first agents do not comprise, or are not, ammonium acetate.

The first agent (e.g., a carboxylate polymer and a sulfonate polymer) is capable of maintaining water solubility upon coordination of the multivalent cation of a second agent. A first agent is capable of maintaining water solubility upon coordination of the multivalent cation of a second agent if the water solubility of the first agent, in the presence of the second agent in an amount or at a concentration sufficient to remove inhibitors, is at least 50% (e.g., at least 60%, 70%, 80% or 90%) of the water solubility in the same solution but without the second agent, or if at least 50% (e.g., at least 60%, 70%, 80% or 90%) of the first agent is not precipitated in the mixture of step (a) (e.g., not in the pellet when centrifuged at a low speed, 100 rpm).

In certain preferred embodiments, the first agent is beta-alanine or guanidine sulfate. In certain embodiments, the first agent is not ammonium acetate.

The final concentration of the first agent in the mixture of step (a) may be in the range of 10 to 500 mM, such as 10 to 50 mM, 50 to 100 mM, 100 to 200 mM, 200 to 300 mM, 300 to 400 mM, 400 to 500 mM, 10 to 100 mM, 10 to 200 mM, 10 to 300 mM, 10 to 400 mM, 50 to 200 mM, 50 to 300 mM, 50 to 400 mM, 50 to 500 mM, 100 to 300 mM, 100 to 400 mM, 100 to 500 mM, 200 to 400 mM, 200 to 500 mM, or 300 to 500 mM, preferably 10 to 200 mM or 25 to 100 mM.

If multiple first agents are present in the mixture of step (a), the total final concentration of the multiple first agents in the mixture of step (a) may be in the range of 10 to 500 mM, such as 10 to 50 mM, 50 to 100 mM, 100 to 200 mM, 200 to 300 mM, 300 to 400 mM, 400 to 500 mM, 10 to 100 mM, 10 to 200 mM, 10 to 300 mM, 10 to 400 mM, 50 to 200 mM, 50 to 300 mM, 50 to 400 mM, 50 to 500 mM, 100 to 300 mM, 100 to 400 mM, 100 to 500 mM, 200 to 400 mM, 200 to 500 mM, or 300 to 500 mM, preferably 10 to 200 mM or 25 to 100 mM. The final concentration of an individual first agent in the mixture of step (a) may be in the range of 1 to 450 mM, such as 1 to 10 mM, 10 to 50 mM, 50 to 100 mM, 100 to 200 mM, 200 to 300 mM, 300 to 400 mM, 400 to 450 mM, 1 to 50 mM, 1 to 100 mM, 1 to 200 mM, 1 to 300 mM, 1 to 400 mM, 10 to 100 mM, 10 to 200 mM, 10 to 300 mM, 10 to 400 mM, 10 to 450 mM, 50 to 200 mM, 50 to 300 mM, 50 to 400 mM, 50 to 450 mM, 100 to 300 mM, 100 to 400 mM, 100 to 450 mM, 200 to 400 mM, 200 to 450 mM, or 300 to 450 mM, preferably 1 to 100 mM or 10 to 80 mM.

The second agent is a multivalent salt and is also referred to as an “inhibitor removing agent.” A “multivalent salt” refers to a salt that contains a cation having a valence of at least two. Exemplary second agents include aluminum ammonium sulfate, aluminum ammonium sulfate dodecahydrate, aluminum chloride, aluminum sulfate, erbium (III) acetate, erbium (III) chloride, holmium chloride, zirconium (IV) chloride, hafnium (IV) chloride, and combinations thereof, preferably aluminum ammonium sulfate, aluminum ammonium sulfate dodecahydrate, aluminum chloride, and combinations thereof. Additional second agents include aluminum ammonium sulfate, aluminum ammonium sulfate dodecahydrate, aluminum potassium sulfate, aluminum chlorohydrate, calcium oxide, iron (III) chloride, iron (II) sulfate, magnesium chloride, and combinations thereof. In certain embodiments, the second agent is not aluminum ammonium sulfate or aluminum ammonium sulfate dodecahydrate.

The final concentration of the second agent in the mixture of step (a) may be in the range of 1 to 150 mM, such as 1 to 5 mM, 5 to 25 mM, 25 to 50 mM, 50 to 75 mM, 75 to 100 mM, 100 to 150 mM, 1 to 25 mM, 1 to 50 mM, 1 to 75 mM, 1 to 100 mM, 1 to 150 mM, 5 to 50 mM, 5 to 75 mM, 5 to 100 mM, 5 to 150 mM, 25 to 75 mM, 25 to 100 mM, 25 to 150 mM, 50 to 100 mM, 50 to 150 mM, 75 to 150 mM, preferably, 5 to 25 mM or 5 to 50 mM.

If multiple second agents are present in the mixture of step (a), the total final concentration of the multiple second agents in the mixture of step (a) may be in the range of 1 to 150 mM, such as 1 to 5 mM, 5 to 25 mM, 25 to 50 mM, 50 to 75 mM, 75 to 100 mM, 100 to 150 mM, 1 to 25 mM, 1 to 50 mM, 1 to 75 mM, 1 to 100 mM, 1 to 150 mM, 5 to 50 mM, 5 to 75 mM, 5 to 100 mM, 5 to 150 mM, 25 to 75 mM, 25 to 100 mM, 25 to 150 mM, 50 to 100 mM, 50 to 150 mM, 75 to 150 mM, preferably, 5 to 25 mM or 5 to 50 mM. The final concentration of an individual second agent in the mixture of step (a) may be in the range of 0.1 to 145 mM, such as 0.1 to 1 mM, 1 to 5 mM, 5 to 25 mM, 25 to 50 mM, 50 to 75 mM, 75 to 100 mM, 100 to 145 mM, 0.1 to 5 mM, 0.1 to 25 mM, 0.1 to 50 mM, 0.1 to 75 mM, 0.1 to 75 mM, 0.1 to 100 mM, 1 to 25 mM, 1 to 50 mM, 1 to 75 mM, 1 to 100 mM, 1 to 145 mM, 5 to 50 mM, 5 to 75 mM, 5 to 100 mM, 5 to 145 mM, 25 to 75 mM, 25 to 100 mM, 25 to 145 mM, 50 to 100 mM, 50 to 145 mM, 75 to 145 mM, preferably, 1 to 20 mM or 2 to 40 mM.

Any of the first agents described above may be used in combination with any of the second agents described above in the inhibitor removal process of the method provided herein. For example, beta-alanine may be used as the first agent to be combined with the following second agent: aluminum ammonium sulfate, aluminum ammonium sulfate dodecahydrate, aluminum chloride, aluminum sulfate, erbium (III) acetate, erbium (III) chloride, holmium chloride, zirconium (IV) chloride, hafnium (IV) chloride, aluminum ammonium sulfate, aluminum ammonium sulfate dodecahydrate, aluminum potassium sulfate, aluminum chlorohydrate, calcium oxide, iron (III) chloride, iron (II) sulfate, magnesium chloride, or a combination thereof, preferably aluminum ammonium sulfate, aluminum ammonium sulfate dodecahydrate, aluminum chloride, or a combination thereof. Similarly, guanidine sulfate may be used as the first agent to be combined with the following second agent: aluminum ammonium sulfate, aluminum ammonium sulfate dodecahydrate, aluminum chloride, aluminum sulfate, erbium (III) acetate, erbium (III) chloride, holmium chloride, zirconium (IV) chloride, hafnium (IV) chloride, aluminum ammonium sulfate, aluminum ammonium sulfate dodecahydrate, aluminum potassium sulfate, aluminum chlorohydrate, calcium oxide, iron (III) chloride, iron (II) sulfate, magnesium chloride, or a combination thereof, preferably aluminum ammonium sulfate, aluminum ammonium sulfate dodecahydrate, aluminum chloride, or a combination thereof. In addition, any two or more of the first agents described above may be used in combination with any of the second agents described above in the inhibitor removal process of the method provided herein; any of the first agents described above may be used in combination with any two or more of the second agents described above in the inhibitor removal process of the method provided herein; and any two or more of the first agents described above may be used in combination with any two or more of the second agents described above in the inhibitor removal process of the method provided herein.

Preferred combinations of the first agent and the second agent include: beta-alanine as the first agent and aluminum ammonium sulfate or aluminum ammonium sulfate dodecahydrate as the second agent, beta-alanine as the first agent and aluminum chloride as the second agent, guanidine sulfate as the first agent and aluminum ammonium sulfate or aluminum ammonium sulfate dodecahydrate as the second agent, guanidine sulfate as the first agent and aluminum chloride as the second agent, histidine as the first agent and aluminum ammonium sulfate or aluminum ammonium sulfate dodecahydrate as the second agent, histidine as the first agent and aluminum chloride as the second agent, glycine as the first agent and aluminum ammonium sulfate or aluminum ammonium sulfate dodecahydrate as the second agent, and glycine as the first agent and aluminum chloride as the second agent.

In step (a), a sample, a lysate of the sample, a supernatant of the lysate or a portion of the sample, the lysate or the supernatant (collectively referred to as “sample material”) may be contacted with the one or more first agents and then with the one or more second agents. In such embodiments, preferably, no separation of solid and liquid phases occurs between contacting the sample material with the one or more first agents and contacting the resulting mixture with the one or more second agents. In other words, preferably, the mixture resulting from contacting with the one or more first agents is not centrifuged, filtrated, precipitated, or otherwise treated to generate a supernatant to be further mixed with the one or more second agents.

Alternatively but less preferably, a sample material may be contacted with the one or more second agents and then with the one or more first agents. In such embodiments, the mixture of the sample material and the one or more second agents is preferably not centrifuged, filtrated, precipitated, or otherwise treated to generate a supernatant to be further mixed with the one or more first agents.

Preferably, a sample material is contacted with the one or more first agents and the one or more second agents at the same time. For example, the sample material may be mixed with a composition (e.g., a solution) that comprises the one or more first agents and the one or more second agents. The concentrations of the one or more first agents and the one or more second agents as well as exemplary preferred solutions are described in detail in the “Compositions” section below.

The mixture of step (a) is centrifuged, filtrated, precipitated, or otherwise treated in step (b) to separate its solid phase from its liquid phase, wherein the one or more first agents are primarily (more than 50%, such as more than 60%, preferably 70% or 80%, or more preferably 90%) in the liquid phase, and wherein the one or more second agents are primarily (more than 50%) in the solid phase. The one or more second agents form complexes with inhibitors and other contaminating materials from the sample, which complexes are precipitated out or otherwise removed from the liquid phase in step (b).

In certain embodiments, more than 60%, 70%, or 80%, preferably more than 90%, or more preferably more than 95% of the one or more second agents is removed from the liquid phase in step (b).

As used herein, the term “inhibitor” refers to any substance that interferes with a reaction involving proteins, DNA and/or RNA isolated from a sample, and has a detrimental effect on protein, DNA and/or RNA manipulation. Inhibitors include, for example, inhibitors of an enzymatic reaction that uses DNA or RNA as a substrate, a contaminant that disrupts hybridization of DNA or RNA, and inhibitors that affect activities of isolated proteins.

Depending on the types of samples, inhibitors may vary. For example, inhibitors in stool samples include haemoglobin and the metabolites thereof, bilirubin, bile acids and bile acid derivatives, undigested or partially digested fiber, or undigested or partially digested food, and polysaccharides.

Inhibitors from environmental samples like soil samples include humic substances formed when microbes degrade plant residues and are stabilized to degradation by covalent binding of their reactive sites to metal ions and clay minerals. They comprise polycyclic aromatics to which saccharides, peptides, and phenols are attached. The predominant types of humic substances in soils are humic acids and fulvic acids. Additional humic substances include humic polymers and humin.

Additional exemplary inhibitors include chitin, decomposing plant materials, organic compounds from compost, phenolics, phenolic polymers or oligomers, polyphenol, polysaccharides, and tannin.

The method provided herein is capable of substantially removing one or more inhibitors from a sample. An inhibitor is substantially removed if 20% or less, preferably 18% or less, 15% or less, 13% or less or 10% or less, more preferably 5% or less, 3% or less, 2% or less or 1% or less of the inhibitor from the sample remains in the liquid phase after separating the mixture that comprises the sample material, optionally a lytic reagent, and the first and second agents into a solid phase and a liquid phase.

In certain embodiments, an inhibitor inhibits PCR amplification of isolated nucleic acids and is referred to as “a PCR inhibitor.” “PCR amplification” as used herein includes various types of PCR reactions, such as qPCR and RT-PCR. The removal of such an inhibitor by a particular inhibitor removal process may be evaluated by comparing certain features (e.g., Ct values) of PCR reactions using nucleic acids isolated with the inhibitor removal process with PCR reactions using nucleic acids isolated without the inhibitor removal process. The degree of reduction in Ct values between the PCR reactions may indicate the effectiveness of the inhibitor removal process in depleting PCR inhibitor(s).

### Biomolecule Isolation

Protein Isolation

The liquid phase generated in step (b) is used to separate and preferably also isolate proteins in step (c). For example, proteins may be precipitated out of the liquid phase by adding a protein-precipitating agent, such as trichloroacetic acid (TCA). Precipitated protein preparation may be pelleted using centrifugation, washed with acetone or other organic solvent to remove residual TCA and/or other substances, and resuspended in an appropriate buffer for proteins.

Proteins may also be isolated by ion exchange chromatography, gel filtration or affinity chromatography. For example, proteins in the liquid phase from step (b) may bind to a protein-binding solid support (e.g., a silica spin filter membrane, a silica spin column, silica-coated magnetic beads, diatomaceous earth, and finely divided suspensions of silica particles), washed using a protein wash solution (e.g., a solution containing ethanol), and subsequently eluted from the solid support using a protein elution solution (e.g., a HEPES (4-(2-hydroxyethyl)-1-piperazineethanesulfonic acid) buffer containing a detergent).

A protein binding solution may be used to facilitate or strengthen the binding of proteins to a protein-binding solid support. The binding solution may comprise a buffer solution (e.g., citrate buffer) and a salt (e.g., NaCl). The final concentration of the salt in the binding mixture may be in the range of 1 to 5M, such as 2 to 3M. The pH is preferably acidic, such as 2 to 5 or 3 to 4.

The amount or concentration of isolated proteins may be measured by a method known in the art, such as spectroscopic methods (see e.g., Brewer et al., J. Biol. Chem. 245:4232, 1970; Pace et al., Protein Sci. 4: 2411, 1995) and colorimetric assays such as Lowry method and Bradford method. The purity of isolated proteins (e.g., total proteins) may be measured by assessing the quantity of particular types of impurities using a method known in the art. The integrity of isolated proteins may be analyzed by a method in the art, such as gel electrophoresis.

Isolated proteins may be analyzed with or without further purification by, for example, 1-dimensional polyacrylamide gel electrophoresis (1D PAGE), mass spectrometry following in-gel trypsin digestion, 2-dimensional polyacrylamide gel electrophoresis (2D PAGE), ELISA-type assays for assessment of native protein activity, other enzyme-based assays, western blotting, amino acid sequencing, and antibody production (e.g., injecting proteins into animals or generating monoclonal antibodies).

Nucleic Acid Isolation

In preferred embodiments, the method provided herein is also useful in separating and optionally also isolating nucleic acids in addition to proteins from a sample. The term “nucleic acid” as used herein include single- or double-stranded nucleic acids and can be any DNA (e.g., genomic DNA, plasmid DNA, bacterial DNA, yeast DNA, viral DNA, plastid DNA, cosmid DNA, and mitochondrial DNA) or any RNA (e.g., rRNA, tRNA, mRNA, and snRNA).

Nucleic acid isolation may be performed in parallel with protein isolation. In other words, the liquid phase obtained in step (b) may be divided into at least two portions: One portion is used for nucleic acid isolation while another portion is used for protein isolation.

Alternatively, nucleic acid isolation may be performed sequentially with protein isolation. In such embodiments, nucleic acids and proteins are isolated from the same liquid phase or the same portion of the liquid phase sequentially, rather than from different portions of the liquid phase.

Nucleic Acid Isolation in Parallel with Protein Isolation

In the embodiments wherein nucleic acid isolation is performed in parallel with protein isolation, any methods suitable for isolating DNA, RNA, or both DNA and RNA from a solution may be used. Preferably, a nucleic acid-binding solid support is used in nucleic acid isolation. Exemplary solid support includes silica matrices, glass particles, diatomaceous earth, magnetic beads, nitrocellulose, nylon, and anion-exchange materials. The solid support may be in the form of loose particles, filters, membranes, fibers or fabrics, or lattices, and contained in a vessel, including tubes, columns, and preferably a spin column.

To facilitate or strengthen binding of nucleic acids to a solid support, a binding solution may be used. The binding solution may be added during sample lysis (e.g., after mechanical disruption of the sample in the presence of a lytic reagent) before contacting the sample material with a first agent and a second agent during the inhibitor removal process. Alternatively, the binding solution may be added to the liquid phase obtained after the inhibitor removal process.

Exemplary DNA binding solution may comprise a chaotropic agent (e.g., GuSCN or GuHCl), an alcohol (e.g., ethanol or isopropanol), or both. It may further comprise a buffer substance, such as Tris HCl.

In the embodiments where both DNA and RNA are separated and optionally isolated in addition to proteins from a sample, DNA separation and optional isolation and RNA separation and optional isolation may be performed in parallel. In other words, the liquid phase of step (b) is divided into at least three portions: one for DNA isolation, one for RNA isolation, and one for protein isolation. Preferably, DNA and RNA are separated and optionally isolated sequentially. Put differently, the liquid phase of step (b) may be divided into two portions: one for sequentially separating and optionally isolating DNA and RNA, and the other for protein separation and optional isolation.

Methods for sequentially isolating DNA and RNA are known (see e.g., U.S. Pat. No. 8,889,393, WO 2004/108925, Triant and Whitehead, Journal of Heredity 100:246-50, 2009). Preferably, a solid support for binding DNA and a solid support for binding RNA are used. The solid support for binding DNA may be identical to or different from the solid support for binding RNA. As used herein, the term “identical” means that two solid supports (e.g., two spin columns) have the same structural and functional characteristics and are of the same kind. When two solid supports identical to each other are used for DNA and RNA isolation, differential binding of DNA and RNA to the solid supports may be achieved by adjusting the component(s) and/or their concentration(s) of binding mixtures. For example, a silica spin column may be used to bind DNA first while the flow through may be mixed with ethanol, and the resulting mixture is applied to a second silica spin column to bind RNA.

After binding to a solid phase, DNA or RNA bound to the solid phase may be washed, and subsequently optionally eluted from the solid phase. It is also possible to not elute the DNA and/or the RNA from the solid phase and apply any downstream application directly to the still bound nucleic acids. Moreover, it is also possible to elute only one of the nucleic acids if not both actually are desired, e.g., elute only DNA and discard the solid phase with the bound RNA or elute only RNA and discard the solid phase with the bound DNA if separate solid phases are used for binding RNA and DNA. DNA wash solution may comprise a chaotropic agent (e.g., GuHCl), an alcohol (e.g., ethanol, isopropanol), or both. It may further comprise a buffer substance (e.g., Tris HCl), a chelating agent (e.g., EDTA (ethylenediaminetetraacetic acid)), and/or a salt (e.g., NaCl). DNA elution solution may be a buffer (e.g., a Tris buffer) or water.

RNA binding solution may comprise alcohol (e.g., ethanol, isopropanol) and optionally another organic solvent (e.g., acetone). RNA wash solution may comprise one or more of the following: a buffer substance (e.g., Tris HCl and Tris base), a chelating agent (e.g., EDTA), an alcohol, and a salt (e.g., NaCl). RNA may be eluted from a solid support using DEPC-treated or other RNase-free water.

Nucleic Acid Separation and/or Isolation Sequentially with Protein Isolation

In the embodiments wherein nucleic acid separation and/or isolation and protein isolation are performed sequentially, the liquid phase of step (b) is treated to generate different fractions that contain nucleic acids and proteins separately. Preferably, all of the three major biomolecules, DNA, RNA and proteins are sequentially separated and optionally isolated.

Preferably, the method for sequentially separating and optionally isolating DNA, RNA and proteins from a sample, comprises:

(a) contacting a sample, a lysate of the sample, a supernatant of the lysate, or a portion of the sample, the lysate or the supernatant with one or more first agents selected from low molecular weight carboxylates, low molecular weight sulfates, carboxylate polymers, sulfonated polymers, or mixtures thereof, and one or more second agents that are multivalent salt(s) to generate a mixture;

(b) separating the mixture of step (a) into a solid phase and a liquid phase, wherein the one or more first agents are primarily in the liquid phase, and wherein the one or more second agents are primarily in the solid phase;

(c) separating and optionally isolating DNA from the liquid phase of step (b), comprising:
(1) contacting the liquid phase of step (b) with a first solid support under conditions so that DNA in the liquid phase of step (b) binds to the first solid support,
(2) optionally washing the DNA bound to the first solid support in step (c)(1), and
(3) optionally eluting the DNA optionally washed in step (c)(2) from the first solid support;

(d) separating and optionally isolating RNA from the flow through obtained from step (c)(1), comprising:
(1) contacting the flow through obtained from step (c)(1) with a second solid support under conditions so that RNA in the flow through obtained from step (c)(1) binds to the second solid support,
(2) optionally washing the RNA bound to the second solid support in step (d)(1), and
(3) optionally eluting the RNA optionally washed in step (d)(2) from the second solid support, and

(e) separating and optionally isolating protein from the flow through obtained from step (d)(1), comprising:
(1) contacting the flow through obtained from step (d)(1) with a third solid support under conditions so that proteins in the flow through obtained from step (d)(1) bind to the second solid support,
(2) optionally washing the proteins bound to the third solid support in step (e)(1), and
(3) optionally eluting the protein optionally washed in step (e)(2) from the third solid support.

The description provided above about the method of the present disclosure in general is also applicable to the steps (e.g., steps (a) and (b)) of the above preferred method and to the reagents or agents (e.g., the lytic reagents, the first agent, and the second agent) and their concentrations used in the above preferred method.

In certain embodiments, after a sample is lyzed, the resulting lysate or a portion thereof may be optionally centrifuged to obtain supernatant. The lysate, the supernatant, or a portion of the lysate and the supernatant is then contacted with the one or more first and second agents during the inhibitor removal process. The sample lysis is preferably performed using a lytic reagent that comprises one or more phosphates and one or more relatively mild chaotropic agents in combination with mechanical disruption as described above.

Two or all of the first solid support, the second solid support, and the third solid support may be identical to or different from each other. In the embodiments where they are identical, the binding conditions (e.g., binding mixtures) primarily determine which biomolecules bind to the solid supports.

An exemplary method according to the above-described preferred embodiment is described in more detail in Example 7 below. Briefly, a sample is lyzed by a lytic reagent in combination with bead beating to efficiently solubilize nucleic acids and proteins from the sample. The lysate is mixed with a DNA binding solution. DNA is bound to a silica spin column and the flow through containing RNA and proteins is then combined with a solution that binds total RNA on a second silica spin column. The final flow through, containing denatured proteins, is combined with another buffer to immobilize the proteins onto a third and final silica spin column. Each spin column containing either immobilized nucleic acids or proteins is then washed and the immobilized biomolecules are eluted.

The yields and purity of isolated nucleic acids may be determined using the NANODROP® ND1000 spectrophotometer (NanoDrop Technologies Inc., Wilmington, Del.), the QUBIT™ dsDNA HS Assay Kit (Q32854) as well as the QUBIT™ dsDNA Br Assay Kit (Q32853) on the QUBIT™ Fluorometer (Invitrogen Co., Carlsbad, Calif.), QUANT-IT™ High-Sensitivity dsDNA Assay Kit (ThermoFisher), a QUANT-IT™ RNA Assay Kit. The yield of DNA may be different measured by a spectrophotometer and a fluorometer. DNA concentration measured by the NANODROP® spectrophotometer has been observed to be higher than that measured by the QUBIT™ fluorometer in some cases.

Purity of isolated DNA and RNA may be assessed by measuring the A260/A280 nm ratio, the A260/A230 nm ratio, and A340 using for example the NANODROP® NDIOOO spectrophotometer (NanoDrop Technologies Inc., Wilmington, Del.).

Pure DNA and RNA have A260/A280 nm ratios of 1.8 and 2.0, respectively. If there is significant contamination with proteins or phenol, the A260/A280 ratio will be less than the values given above.

The A260/A230 nm ratio is a measure of contaminants that absorb at 230 nm. Pure DNA and RNA have A260/A230 nm ratios of 2.0-2.2. Significant absorption at 230 nm indicates contamination by phenolate ion, thiocyanates, and other organic compounds.

Absorption at 340 nm (i.e., A340) is usually caused by light scattering and indicates the presence of particulate matter.

DNA isolated according to a method provided herein may have one or more of the following features:

(1) Its A260/A280 is in the range of 1.6 to 2.0, preferably 1.7 to 1.9, and more preferably 1.75 to 1.85.

(2) Its A260/A230 is in the range of 1.0 to 2.5, preferably 1.5 to 2.2.

(3) Its A340 is in the range of 0 to 0.15, preferably 0 to 0.1, more preferably 0 to 0.05.

RNA isolated according to a method provided herein may have one or more of the following features:

(1) Its A260/A280 is in the range of 1.8 to 2.2, preferably 1.9 to 2.1, and more preferably 1.95 to 2.05.

(2) Its A260/A230 is in the range of 1.0 to 2.5, preferably 1.5 to 2.2.

(3) Its A340 is in the range of 0 to 0.15, preferably 0 to 0.1, more preferably 0 to 0.05.

The integrity of isolated DNA may be assessed by visualizing extracted DNA on an agarose gel. The integrity of isolated RNA may also be assessed by visualizing extracted RNA using gel electrophoresis.

The isolated DNA may be analyzed or used in any application, including PCR, qPCR, RT-PCR, rolling circle replication, ligase-chain reaction, sequencing (e.g., next generation sequencing, southern, dot, and slot blot analyses, DNA methylation analysis, mass spectrometry, and electrophoresis.

The isolated RNA may be analyzed or used in any application, such as RT-PCR, real-time RT-PCR, differential display, cDNA synthesis, Northern, dot, and slot blot analyses, and microarray analysis.

### Compositions

In another aspect, the present disclosure provides a composition useful in removing inhibitors during protein (and optionally nucleic acid) isolation from a sample. The composition comprises, consists essentially of, or consists of one or more first agents selected from low molecular weight carboxylates, low molecular weight sulfates, carboxylate polymers, sulfonated polymers, or mixtures thereof, one or more second agents that are multivalent salt(s), and optionally water.

The first agent and the second agent are described above in connection with methods for isolating proteins (and optionally nucleic acids) from a sample.

In certain compositions, the one or more first agents are selected from amino acids; salts of short chain fatty acids, such as sodium butyrate; sodium polystyrene sulfonate; sodium polyacrylic acid; preferably ammonium glycolate, sulfoacetic acid, ammonium formate, and cesium acetate, preferably, beta-alanine, guanidine sulfate, histidine, glycine, and combinations thereof, and the one or more second agents are selected from aluminum sulfate, erbium (III) acetate, erbium (III) chloride, holmium chloride, zirconium (IV) chloride, hafnium (IV) chloride, aluminum ammonium sulfate, aluminum ammonium sulfate dodecahydrate, aluminum potassium sulfate, aluminum chlorohydrate, calcium oxide, iron (III) chloride, iron (II) sulfate, magnesium chloride, and combinations thereof, preferably aluminum ammonium sulfate, aluminum ammonium sulfate dodecahydrate, aluminum chloride, and combinations thereof.

In certain preferred compositions, the one or more first agents are selected from amino acids; salts of short chain fatty acids, such as sodium butyrate; sodium polystyrene sulfonate; sodium polyacrylic acid; preferably beta-alanine, guanidine sulfate, histidine, glycine, and combinations thereof, and the one or more second agents are selected from aluminum sulfate, erbium (III) acetate, erbium (III) chloride, holmium chloride, zirconium (IV) chloride, hafnium (IV) chloride, aluminum ammonium sulfate, aluminum ammonium sulfate dodecahydrate, aluminum potassium sulfate, aluminum chlorohydrate, calcium oxide, iron (III) chloride, iron (II) sulfate, magnesium chloride, aluminum ammonium sulfate, aluminum ammonium sulfate dodecahydrate, aluminum chloride, and combinations thereof.

In certain other embodiments, the one or more first agents are selected from amino acids; salts of short chain fatty acids, such as sodium butyrate; sodium polystyrene sulfonate; sodium polyacrylic acid; preferably ammonium sulfate, ammonium glycolate, sulfoacetic acid, ammonium formate, sodium acetate, cesium acetate, and combinations thereof, more preferably, ammonium acetate, beta-alanine, guanidine sulfate, histidine, glycine, and combinations thereof, and the one or more second agents are selected from erbium (III) acetate, erbium (III) chloride, holmium chloride, zirconium (IV) chloride, hafnium (IV) chloride, and combinations thereof, preferably aluminum chloride.

In certain preferred compositions, the one or more first agents are selected from amino acids; salts of short chain fatty acids, such as sodium butyrate; sodium polystyrene sulfonate; sodium polyacrylic acid; preferably ammonium sulfate, ammonium glycolate, sulfoacetic acid, ammonium formate, sodium acetate, cesium acetate, ammonium acetate, beta-alanine, guanidine sulfate, histidine, glycine, and combinations thereof, and the second agent is aluminum chloride.

The composition is preferably an aqueous solution. In such a case, the concentration of the first agent in the solution may be in the range of 0.1 to 1M, such as 0.1 to 0.25M, 0.25 to 0.5M, 0.5 to 0.75M, 0.75 to 1M, 0.1 to 0.5M, 0.1 to 0.75M, 0.25 to 0.75M, 0.25 to 1M, or 0.5 to 1M, preferably 0.1 to 0.75M.

If multiple first agents are present in the solution, the total concentration of the first agents in combination in the solution may be in the range of 0.1 to 1M, such as 0.1 to 0.25M, 0.25 to 0.5M, 0.5 to 0.75M, 0.75 to 1M, 0.1 to 0.5M, 0.1 to 0.75M, 0.25 to 0.75M, 0.25 to 1M, or 0.5 to 1M, preferably 0.1 to 0.75M. The concentration of an individual first agent in the solution may be in the range of 0.01 to 0.95M, such as 0.01 to 0.1M, 0.1 to 0.25M, 0.25 to 0.5M, 0.5 to 0.75M, 0.75 to 1M, 0.01 to 0.25M, 0.01 to 0.5M, 0.01 to 0.75M, 0.1 to 0.5M, 0.1 to 0.75M, 0.25 to 0.75M, 0.1 to 0.95M, 0.25 to 0.95M, or 0.5 to 0.95M, preferably 0.05 to 0.75M.

The concentration of the second agent in the solution may be in the range of 10 to 500 mM, such as 10 to 100 mM, 100 to 200 mM, 200 to 300 mM, 300 to 400 mM, 400 to 500 mM, 10 to 200 mM, 10 to 300 mM, 10 to 400 mM, 100 to 300 mM, 100 to 400 mM, 100 to 500 mM, 200 to 400 mM, 200 to 500 mM, 300 to 500 mM, preferably 10 to 200 mM, 10 to 500 mM, 50 to 200 mM, 50 to 500 mM, or 75 to 150 mM.

If multiple second agents are present in the solution, the total concentration of the second agents in combination in the solution may be in the range of 10 to 500 mM, such as 10 to 100 mM, 100 to 200 mM, 200 to 300 mM, 300 to 400 mM, 400 to 500 mM, 10 to 200 mM, 10 to 300 mM, 10 to 400 mM, 100 to 300 mM, 100 to 400 mM, 100 to 500 mM, 200 to 400 mM, 200 to 500 mM, 300 to 500 mM, preferably 10 to 200 mM, 10 to 500 mM, 50 to 200 mM, 50 to 500 mM, or 75 to 150 mM. The concentration of an individual second agent in the solution may be in the range of 1 to 450 mM, such as 1 to 10 mM, 10 to 100 mM, 100 to 200 mM, 200 to 300 mM, 300 to 400 mM, 400 to 450 mM, 1 to 100 mM, 1 to 200 mM, 1 to 300 mM, 1 to 400 mM, 10 to 200 mM, 10 to 300 mM, 10 to 400 mM, 10 to 450 mM, 100 to 300 mM, 100 to 400 mM, 100 to 450 mM, 200 to 400 mM, 200 to 450 mM, 300 to 450 mM, preferably 1 to 150 mM, 10 to 450 mM, 50 to 150 mM, 50 to 450 mM, or 10 to 150 mM.

Exemplary preferred solutions that comprise the first agent and the second agent include:

(1) a solution containing 0.2 to 0.8M (e.g., 0.25M, 0.5M, or 0.75M) guanidine sulfate and 20 to 200 mM (e.g., 50 mM, 100 mM, or 150 mM) aluminum ammonium sulfate or aluminum ammonium sulfate dodecahydrate;

(2) a solution containing 0.25 to 1M (e.g., 0.5M or 0.75M) beta-alanine and 20 to 200 mM (e.g., 50 mM, 100 mM, or 150 mM) aluminum ammonium sulfate or aluminum ammonium sulfate dodecahydrate;

(3) a solution containing 0.25 to 1M (e.g., 0.25M, 0.5M, 0.75M, or 1M) glycine and 20 to 200 mM (e.g., 50 mM, 100 mM, or 150 mM) aluminum ammonium sulfate or aluminum ammonium sulfate dodecahydrate;

(4) a solution containing 0.25 to 1M (e.g., 0.25M, 0.5M, or 0.75M) histidine and 20 to 200 mM (e.g., 50 mM, 100 mM, or 150 mM) aluminum ammonium sulfate or aluminum ammonium sulfate dodecahydrate;

(5) a solution containing 0.2 to 0.8M (e.g., 0.25M, 0.5M, or 0.75M) guanidine sulfate and 20 to 200 mM (e.g., 50 mM, 100 mM, or 150 mM) aluminum chloride;

(6) a solution containing 0.25 to 1M (e.g., 0.5M or 0.75M) beta-alanine and 20 to 200 mM (e.g., 50 mM, 100 mM, or 150 mM) aluminum chloride;

(7) a solution containing 0.25 to 1M (e.g., 0.25M, 0.5M, 0.75M, or 1M) glycine and 20 to 200 mM (e.g., 50 mM, 100 mM, or 150 mM) aluminum chloride; and

(8) a solution containing 0.25 to 1M (e.g., 0.25M, 0.5M, or 0.75M) histidine and 20 to 200 mM (e.g., 50 mM, 100 mM, or 150 mM) aluminum chloride.

Alternatively, the composition may be in solid form. In such a case, the composition comprises, consists essentially of, or consists of the one or more first agents and the one or more second agents so that when an appropriate amount of water is added to the composition, the resulting solution has the concentrations of the one or more first agents and the one or more second agents as described above in the case where the composition is already a solution. The water that is added may also result from the water in the sample, i.e., the combination of salts may be added directly to the aqueous sample material.

In a related aspect, the present disclosure provides the use of the above-described compositions in isolating proteins (and optionally nucleic acids) from a sample.

### Kits

In another aspect, the present disclosure provides a kit for isolating proteins (and optionally nucleic acids) from a sample. The kit comprises:

(a) the composition disclosed herein, or

(b) the one or more first agents and the one or more second agents of the composition disclosed herein provided separately.

The kit may further comprise one or more of the following components:

a lytic reagent (preferably a lytic reagent comprising, consisting essentially of, or consisting of one or more phosphates and one or more relatively mild chaotropic agents as described above),

a homogenizing material (i.e., a substance useful in homogenizing a sample such as beads, preferably high density beads) for mechanically disrupting a sample,

a protein binding solid support,

a protein binding solution,

a protein wash solution,

a protein elution solution,

a nucleic acid-binding solid support,

a DNA binding solution,

a DNA wash solution,

a DNA elution solution,

a RNA binding solution,

a RNA wash solution,

a RNA elution solution, and

one or more vessels or containers (e.g., collection tubes).

The above kit components or optional kit components are as described in the “Methods” and “Compositions” sections above.

In a related aspect, the present disclosure provides the use of the above-described kit in isolating proteins (and optionally nucleic acids) from a sample.

### EXAMPLES

The following reagents are referred to in the examples below:
Lytic Reagent I: 1M NaSCN, 0.2M Na₂HPO₄.
Lytic Reagent II: 0.09 M Guanidine Thiocyanate, 0.13 M Na₂HPO₄, 0.006 M NaCl, 1.76M ammonium acetate, 0.25% SDS, 0.10% Antifoam A.
DNA binding solution: containing a chaotropic agent.
DNA wash solution I: containing a chaotropic agent, buffer, and isopropanol, and ethanol.
DNA wash solution II: containing buffer, a chelating agent, a salt, and ethanol.
DNA elution solution: containing buffer with a slightly basic pH.
RNA binding solution I: containing acetone and ethanol.
RNA binding solution II: containing isopropanol.
RNA wash solution: containing buffer, a chelating agent, a salt and isopropanol.
Protein binding solution: containing a salt and buffer with an acidic pH.
Protein wash solution: containing ethanol.

Protein elution solution: buffer with slightly basic pH and detergent.

### Example 1

### Effects of Lytic Reagents with or without Inhibitor Removal on DNA Isolation from Stool Samples

This example examines the effects of different lytic reagents with or without inhibitor removal on DNA isolation from stool samples.

Four different experiments (A, B, C, and D) were performed as shown in the table below. A and B used an exemplary lytic reagent (“lytic reagent I”) of the present disclosure while C and D used an existing lytic reagent (“lytic reagent II”). A and C did not perform inhibitor removal while B and D did.

| | | A | B | C | D |
|---|---|---|---|---|---|
| Input | 0.2 g frozen dog stool | X | X | X | X |
| Lysis, 650 μl | Lytic reagent I | X | X | | |
| | Lytic reagent II | | | X | X |
| | 100 ul phenol | | | X | X |
| | 6.5 μl beta- | X | X | X | X |
| | mercaptoethanol | | | | |
| | (beta-ME) | | | | |
| | 6.5 μl Protease Inhibitors | X | X | | |
| DNA Bind (350 μl) | DNA binding solution | X | X | | |
| Inhibitor Removal (150 μl) | 52 mM AASD | | X | | |
| | 0.12M AASD | | | | X |
| DNA Bind (350 μl) | DNA binding solution | | | X | X |
| DNA Wash | DNA wash solution I | X | X | X | X |
| | DNA wash solution II | X | X | X | X |
| DNA Elute | DNA elute solution | X | X | X | X |

The yields and purity of isolated DNA are shown in the tables below:

Quant-iT dsDNA

| | | |
|---|---|---|
| Sample | Sample Concentration | Average |

| | | | |
|---|---|---|---|
| A | 130 | ug/mL | |
| A | 111 | ug/mL | |
| A | 131 | ug/mL | |
| A | 128 | ug/mL | |
| A | 116 | ug/mL | 123.2 |
| B | 85 | ug/mL | |
| B | 78.6 | ug/mL | |
| B | 79.7 | ug/mL | |
| B | 93 | ug/mL | |
| B | 85.3 | ug/mL | 84.32 |
| C | 68.6 | ug/mL | |
| C | 69.4 | ug/mL | |
| C | 66.5 | ug/mL | |
| C | 60.6 | ug/mL | |
| C | 61.4 | ug/mL | 65.3 |
| D | 38.6 | ug/mL | |
| D | 39.1 | ug/mL | |
| D | 40.6 | ug/mL | |
| D | 40.9 | ug/mL | |
| D | 41.8 | ug/mL | 40.2 |

The above results were obtained using the QUANT-IT™ dsDNA Assay Kit (ThermoFisher Scientific) according to the provider's instructions.

### NanoDrop

| Sample | DNA(ng/uL) | A260/A280 | A260/A230 | A340 |
|---|---|---|---|---|
| A | 167.176 | 1.826 | 1.016 | 0.096 |
| A | 152.183 | 1.821 | 1.153 | 0.109 |
| A | 166.272 | 1.81 | 1.431 | 0.125 |
| A | 161.637 | 1.822 | 1.393 | 0.091 |
| A | 160.319 | 1.816 | 1.406 | 0.109 |
| B | 111.448 | 1.832 | 0.799 | 0.046 |
| B | 110.381 | 1.831 | 0.64 | 0.074 |
| B | 112.97 | 1.81 | 1.734 | 0.051 |
| B | 113.903 | 1.843 | 1.53 | 0.075 |
| B | 108.207 | 1.828 | 0.956 | 0.079 |
| C | 101.056 | 1.782 | 0.779 | 0.077 |
| C | 97.665 | 1.801 | 1.447 | 0.104 |
| C | 98.104 | 1.788 | 1.666 | 0.113 |
| C | 97.556 | 1.793 | 1.267 | 0.078 |
| C | 94.579 | 1.799 | 1.429 | 0.121 |
| D | 58.715 | 1.767 | 0.314 | −0.321 |
| D | 55.321 | 1.802 | 0.593 | 0.039 |
| D | 60.959 | 1.753 | 0.226 | 0.051 |
| D | 57.62 | 1.799 | 0.324 | 0.07 |
| D | 58.641 | 1.782 | 0.662 | 0.054 |

The above results were obtained using THERMOSCIENTIFIC™ NANODROP™ ND-1000 spectrophotomer (ThermoFisher Scientific) according to the provider's instructions.

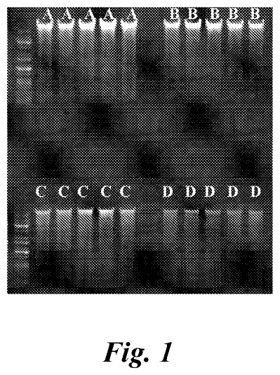

The gel electrophoresis of the isolated DNA is shown in FIG. 1.

The results show that compared to lytic reagent II, lytic reagent I extracted and solubilized much more DNA. Without inhibitor removal, the difference in DNA yield between the two lysis methods was 47%. With inhibitor removal, the difference was 52%.

### Example 2

### Effects of Titration of Ammonium Acetate on DNA, RNA and Protein Isolation from Stool Samples

This example examines the effects of various concentrations of ammonium acetate on DNA, RNA and protein isolation.

Dog stool was previously collected and immediately frozen. The aliquot used for this experiment had been thawed once. Bead beating was in the mixed zirconium bead tubes (1.2 g 0.1 mm+1.2 g 0.5 mm) for 10 minutes on the vortex at maximum setting. After lysis and the addition of DNA binding solution, all the supernatants were pooled. About 800 μl was recovered from each tube, but 750 μl was re-aliquoted for the inhibitor removal step. All the concentrations of NH₄OAc are the concentrations after being combined with aluminum ammonium sulfate dodecahydrate (AASD).

| | | A | B | C | D | E | F |
|---|---|---|---|---|---|---|---|
| Input | 0.2 g frozen dog stool | X | X | X | X | X | X |
| Lysis, 650 μl | 1MNaSCN + 0.2MNa₂HPO₄ | X | X | X | X | X | X |
| | 6.5 μl beta-ME | X | X | X | X | X | X |
| | 6.5 μl Protease Inhibitors | X | X | X | X | X | X |
| DNA Bind (350 μl) | DNA binding solution | X | X | X | X | X | X |
| Inhibitor Removal (150 μl) | Water | X | | | | | |
| | 0.06MAASD | | X | X | X | X | X |
| | 3.75MNH₄OAc | | | X | | | |
| | 0.9375M NH₄Oac | | | | X | | |
| | 0.46875M NH₄Oac | | | | | X | |
| | 0.234375M NH₄OAc | | | | | | X |
| DNA Wash | DNA wash solution I | X | X | X | X | X | X |
| | DNA wash solution II | X | X | X | X | X | X |
| DNA Elute | DNA elution solution | X | X | X | X | X | X |
| RNA Bind (750 μl) | RNA binding solution I | X | X | X | X | X | X |
| RNA Wash | RNA wash solution | X | X | X | X | X | X |
| | Ethanol | X | X | X | X | X | X |
| RNA Elute | RNase-free water | X | X | X | X | X | X |
| Protein Bind (1400 μl) | Protein binding solution | X | X | X | X | X | X |
| Protein Wash | Protein wash solution | X | X | X | X | X | X |
| Protein Elute | Protein elution solution | X | X | X | X | X | X |

The yields and purity of isolated nucleic acids are shown in the tables below:

### NanoDrop

| Sample | DNA(ng/uL) | A260/A280 | A260/A230 | A340 |
|---|---|---|---|---|
| A | 104.416 | 1.825 | 0.438 | 0.007 |
| A | 95.522 | 1.831 | 1.109 | 0.043 |
| B | 85.73 | 1.839 | 0.517 | 0.025 |
| B | 101.538 | 1.82 | 0.442 | 0.019 |
| C | 53.212 | 1.837 | 0.169 | 0.038 |
| C | 46.831 | 1.862 | 0.568 | −0.039 |
| D | 90.398 | 1.874 | 0.145 | 0.119 |
| D | 95.833 | 1.83 | 0.683 | 0.023 |
| E | 100.071 | 1.823 | 1.028 | 0.021 |
| E | 103.32 | 1.827 | 1.514 | −0.008 |
| F | 103.579 | 1.82 | 0.904 | 0.014 |
| F | 99.634 | 1.974 | 0.497 | 21.406 |
| Sample | RNA(ng/uL) | A260/A280 | A260/A230 | A340 |
| A | 278.767 | 2.052 | 0.986 | 21.997 |
| A | 316.745 | 1.983 | 1.059 | 0.551 |
| B | 344.048 | 1.996 | 1.137 | 0.471 |
| B | 307.839 | 1.993 | 0.971 | 0.407 |
| C | 394.183 | 2.001 | 1.21 | 0.582 |
| C | 389.246 | 2.003 | 1.18 | 0.649 |
| D | 364.583 | 1.995 | 1.163 | 0.991 |
| D | 314.102 | 1.995 | 1.066 | 0.524 |
| E | 363.972 | 1.998 | 0.69 | 0.602 |
| E | 329.715 | 2.009 | 1.024 | 0.502 |
| F | 365.367 | 2.01 | 1.086 | 0.506 |
| F | 335.324 | 2 | 1.095 | 0.426 |

### DNA Qubit

| | | |
|---|---|---|
| Sample | DNA Concentration | Average |

| | | | |
|---|---|---|---|
| A | 69.9 | ug/mL | |
| A | 67.3 | ug/mL | 68.6 |
| B | 59 | ug/mL | |
| B | 65.3 | ug/mL | 62.2 |
| C | 30 | ug/mL | |
| C | 27.3 | ug/mL | 28.7 |
| D | 54.7 | ug/mL | |
| D | 63 | ug/mL | 58.9 |
| E | 70 | ug/mL | |
| E | 71.8 | ug/mL | 70.9 |
| F | 72.6 | ug/mL | |
| F | 79.6 | ug/mL | 76.1 |

The results from the above table were obtained using INVITROGEN™ QUBIT™ fluorometer (Invitrogen) according to the provider's instructions.

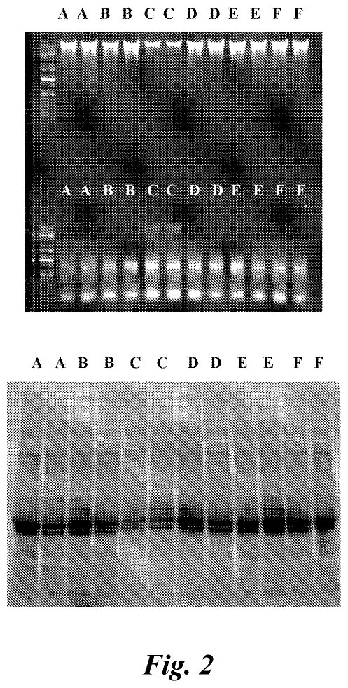

The gel electrophoresis of the isolated DNA, RNA and proteins is shown in FIG. 2, upper, middle and lower panels, respectively.

The results show that 3.75M ammonium acetate caused substantial DNA loss and a reduction in DNA binding. DNA, RNA and protein isolation were all improved to match or exceed the control (3.75M ammonium acetate) when ammonium acetate was used at or lower than 0.9375M. The best yields for all the nucleic acid and protein with A260/A230 at least 1.0 was E (0.234375M ammonium acetate) for DNA and D (0.46875M ammonium acetate) for RNA.

### Example 3

### Effects of Histidine and Glycine on DNA Isolation from Stool Samples

This example examines the effects of histidine and glycine on DNA isolation from stool samples.

Bead beating was in the mixed zirconium bead tubes (1.2 g 0.1 mm+1.2 g 0.5 mm) for 10 minutes on the vortex at maximum setting. After addition of the DNA binding solution, all the samples were pooled, and 750 μl was redistributed into each tube for the inhibitor removal step.

| | | A | B | C | D | E | F | G |
|---|---|---|---|---|---|---|---|---|
| Input | 0.2 g fresh dog stool | X | X | X | X | X | X | X |
| Lysis, 650 μl | 1M NaSCN + 0.2MNa₂HPO₄ | X | X | X | X | X | X | X |
| | 6.5 μl beta-ME | X | X | X | X | X | X | X |
| | 6.5 μl Protease Inhibitors | X | X | X | X | X | X | X |
| DNA Bind (350 μl) | DNA binding solution I | X | X | X | X | X | X | X |
| Inhibitor Removal (150 μl) | Water | X | | | | | | |
| | 0.06M AASD | | X | | | | | |
| | 0.45M histidine + 0.06M AASD | | | X | | | | |
| | 0.23M histidine + 0.06M AASD | | | | X | | | |
| | 0.9M glycine + 0.06M AASD | | | | | X | | |
| | 0.45M glycine + 0.06M AASD | | | | | | X | |
| | 0.23M glycine + 0.06M AASD | | | | | | | X |
| DNA Wash | DNA wash solution I | X | X | X | X | X | X | X |
| | DNA wash solution II | X | X | X | X | X | X | X |
| DNA Elute | DNA elution solution | X | X | X | X | X | X | X |
| RNA Bind (750 μl) | Acetone/ethanol | X | X | X | X | X | X | X |

The yields and purity of isolated DNA are shown in the tables below.

### NanoDrop

| Sample | DNA(ng/uL) | A260/A280 | A260/A230 | A340 |
|---|---|---|---|---|
| A | 81.067 | 1.857 | 1.229 | 0.04 |
| A | 93.186 | 1.859 | 0.452 | −0.06 |
| B | 82.828 | 1.86 | 1.433 | 0.027 |
| B | 70.875 | 1.857 | 0.789 | 0.021 |
| C | 79.299 | 1.838 | 1.119 | 0.018 |
| C | 94.062 | 1.841 | 1.282 | 0.02 |
| D | 76.441 | 1.853 | 1.766 | 0.032 |
| D | 87.506 | 1.849 | 1.099 | 0.031 |
| E | 76.485 | 1.855 | 0.948 | 0.019 |
| E | 94.122 | 1.846 | 0.55 | 0.031 |
| F | 91.097 | 1.862 | 1.035 | 0.029 |
| F | 89.132 | 1.826 | 0.536 | 0.077 |
| G | 93.976 | 1.865 | 0.871 | 0.047 |
| G | 81.342 | 1.872 | 0.818 | 0.048 |

### Quant-iT dsDNA

| | | |
|---|---|---|
| Sample | Sample Concentration | Average |

| | | | |
|---|---|---|---|
| A | 57.6 | ug/mL | |
| A | 61.5 | ug/mL | 59.6 |
| B | 52.7 | ug/mL | |
| B | 54 | ug/mL | 53.4 |
| C | 53.8 | ug/mL | |
| C | 65.9 | ug/mL | 59.9 |
| D | 55.7 | ug/mL | |
| D | 60.4 | ug/mL | 58.1 |
| E | 63.2 | ug/mL | |
| E | 54.3 | ug/mL | 58.8 |
| F | 58.8 | ug/mL | |
| F | 55.1 | ug/mL | 57 |
| G | 67.7 | ug/mL | |
| G | 60.5 | ug/mL | 64.1 |

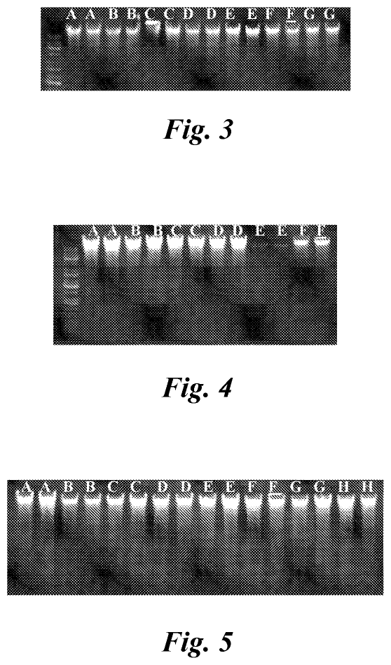

The gel electrophoresis of the isolated DNA is shown in FIG. 3.

The results show that using AASD alone (B) reduced DNA yield compared to the water control (A), while including histidine or glycine in addition of AASD increased DNA yield (C to G) to be similar to the water control (A).

### Example 4

### Effects of Ammonium Sulfate and Ammonium Glycolate on DNA Isolation from Stool Samples

This example examines the effects of ammonium sulfate and ammonium glycolate on DNA isolation from stool samples.

Fresh dog stool was collected and refrigerated for several hours before use. Bead beating was in the mixed zirconium bead tubes (1.2 g 0.1 mm+1.2 g 0.5 mm) for 10 minutes on the vortex at maximum setting. After addition of the DNA binding solution, all the samples were pooled, and 750 μl was redistributed into each tube for the inhibitor removal step.

| | | A | B | C | D | E | F |
|---|---|---|---|---|---|---|---|
| Input | 0.2 g fresh dog stool | X | X | X | X | X | X |
| Lysis, 650 μl | 1M NaSCN + 0.2M Na₂HPO₄ | X | X | X | X | X | X |
| | 6.5 μl beta-ME | X | X | X | X | X | X |
| | 6.5 μl Protease Inhibitors | X | X | X | X | X | X |
| DNA Bind (350 μl) | DNA binding solution I | X | X | X | X | X | X |
| Inhibitor Removal (150 μl) | Water | X | | | | | |
| | 0.06M AASD | | X | | | | |
| | 0.5M (NH₄)₂SO₄ + 0.06M AASD | | | X | | | |
| | 0.25M (NH₄)₂SO₄ + 0.06M AASD | | | | X | | |
| | 0.5M NH₄glycolate + 0.06M AASD | | | | | X | |
| | 0.25M NH₄glycolate + 0.06M AASD | | | | | | X |
| DNA Wash | DNA wash solution I | X | X | X | X | X | X |
| | DNA wash solution II | X | X | X | X | X | X |
| DNA Elute | DNA elution solution | X | X | X | X | X | X |

The yields and purity of isolated DNA are shown in the tables below.

| Sample | DNA(ng/uL) | A260/A280 | A260/A230 | A340 | Average |
|---|---|---|---|---|---|
| A | 154.883 | 1.844 | 2.113 | 0.028 | |
| A | 186.54 | 1.844 | 1.626 | 0.03 | 170.7 |
| B | 144.677 | 1.842 | 1.748 | 0.057 | |
| B | 160.055 | 1.852 | 1.515 | 0.046 | 152.4 |
| C | 138.16 | 1.837 | 2.002 | 0.027 | |
| C | 144.918 | 1.847 | 1.656 | 0.04 | 141.5 |
| D | 124.175 | 1.844 | 0.986 | 0.025 | |
| D | 135.336 | 1.854 | 1.274 | 0.055 | 129.8 |
| E | 18.568 | 1.989 | 0.252 | 0.03 | |
| E | 22.595 | 2.057 | 0.154 | 0.053 | 20.6 |
| F | 94.975 | 1.868 | 0.759 | 0.062 | |
| F | 97.983 | 1.855 | 0.56 | 0.053 | 96.5 |

The gel electrophoresis of the isolated DNA is shown in FIG. 4.

Both ammonium sulfate and ammonium glycolate were soluble in AASD. Ammonium sulfate was better than ammonium glycolate in DNA yields and A260/A280 values. Including 0.5M ammonium sulfate in addition to AASD improved the A260/A230 value compared to using AASD alone to remove inhibitors.

### Example 5

### Effects of Ammonium Formate, Beta-Alanine, and Guanidine Sulfate on DNA Isolation from Stool Samples

This example examines the effects of ammonium formate, betal-alanine, and guanidine sulfate on DNA isolation from stool samples.

Fresh dog stool was collected and refrigerated for several hours before use. Bead beating was in the mixed zirconium bead tubes (1.2 g 0.1 mm+1.2 g 0.5 mm) for 10 minutes on the vortex at maximum setting. After addition of the DNA binding solution, all the samples were pooled, and 750 μl was redistributed into each tube for the inhibitor removal step.

| | | A | B | C | D | E | F | G | H |
|---|---|---|---|---|---|---|---|---|---|
| Input | 0.2 g fresh dog stool | X | X | X | X | X | X | X | X |
| Lysis, 650 μl | 1MNaSCN + 0.2MNa₂HPO₄ | X | X | X | X | X | X | X | X |
| | 6.5 μl beta-ME | X | X | X | X | X | X | X | X |
| | 6.5 μl Protease Inhibitors | X | X | X | X | X | X | X | X |
| DNA Bind (350 μl) | DNA binding solution I | X | X | X | X | X | X | X | X |
| Inhibitor Removal (150 μl) | Water | X | | | | | | | |
| | 0.06M AASD | | X | | | | | | |
| | 0.5M NH₄formate + 0.06M AASD | | | X | | | | | |
| | 0.25M NH₄formate + 0.06M AASD | | | | X | | | | |
| | 0.5M β-Alanine + 0.06M AASD | | | | | X | | | |
| | 0.25M β-Alanine + 0.06M AASD | | | | | | X | | |
| | 0.5M Gu sulfate + 0.06M AASD | | | | | | | X | |
| | 0.25M Gu sulfate + 0.06M AASD | | | | | | | | X |
| DNA Wash | DNA wash solution I | X | X | X | X | X | X | X | X |
| | DNA wash solution II | X | X | X | X | X | X | X | X |
| DNA Elute | DNA elution solution | X | X | X | X | X | X | X | X |

The yields and purity of isolated DNA are shown in the table below.

### NanoDrop

| Sample | DNA(ng/uL) | A260/A280 | A260/A230 | A340 | Average |
|---|---|---|---|---|---|
| A | 120.001 | 1.833 | 1.257 | 0.027 | |
| A | 139.7 | 1.846 | 1.552 | 0.027 | 129.9 |
| B | 84.677 | 1.876 | 1.217 | 0.067 | |
| B | 93.835 | 1.865 | 1.483 | 0.058 | 89.3 |
| C | 104.933 | 1.877 | 0.357 | 0.056 | |
| C | 115.487 | 1.857 | 1.534 | 0.041 | 110.2 |
| D | 111.062 | 1.857 | 0.764 | 0.067 | |
| D | 119.361 | 1.839 | 1.106 | 0.051 | 115.2 |
| E | 118.419 | 1.854 | 1.503 | 0.063 | |
| E | 134.87 | 1.859 | 1.692 | 0.09 | 126.6 |
| F | 109.162 | 1.856 | 1.468 | 0.052 | |
| F | 119.985 | 1.86 | 1.165 | 0.023 | 114.6 |
| G | 126.457 | 1.862 | 1.435 | 0.075 | |
| G | 133.31 | 1.85 | 1.021 | 0.054 | 129.9 |
| H | 127.024 | 1.862 | 0.798 | 0.072 | |
| H | 129.835 | 1.854 | 0.719 | 0.075 | 128.4 |

The gel electrophoresis of the isolated DNA is shown in FIG. 5.

The results show that Ammonium formate, betal-alanine, and guanidine sulfate were soluble in AASD. Using AASD alone (B) reduced DNA yield compared to the water control (A), while including Ammonium formate, betal-alanine, or guanidine sulfate in addition of AASD increased DNA yield (C to H) to be closer to the water control (A).

### Example 6

### Effects of Beta-Alanine on DNA, RNA and Protein Isolation from Stool Samples

This example examines the effects of beta-alanine on DNA, RNA and protein isolation from stool samples

Fresh dog stool was collected and aliquoted immediately into bead tubes. Bead beating was in the mixed zirconium bead tubes (1.2 g 0.1 mm+1.2 g 0.5 mm) for 10 minutes on the vortex at maximum setting. After addition of the DNA binding solution, all the samples were pooled, and 750 μl was redistributed into each tube for the inhibitor removal step.

| | | A | B | C | D | E | F | G |
|---|---|---|---|---|---|---|---|---|
| Input | 0.2 g fresh dog stool | X | X | X | X | X | X | X |
| Lysis, 650 μl | 1MNaSCN + 0.2MNa₂HPO₄ | X | X | X | X | X | X | X |
| | 6.5 μl beta-ME | X | X | X | X | X | X | X |
| | 6.5 μl Protease Inhibitors | X | X | X | X | X | X | X |
| DNA Bind (350 μl) | DNA binding solution I | X | X | X | X | X | X | X |
| Inhibitor Removal (150 μl) | Water | X | | | | | | |
| | 0.06M AASD | | X | | | | | |
| | 0.09M AASD | | | X | | | | |
| | 0.12M AASD | | | | X | | | |
| | 0.5M β-Alanine + 0.06M AASD | | | | | X | | |
| | 0.5M β-Alanine + 0.09M AASD | | | | | | X | |
| | 0.5M β-Alanine + 0.12M AASD | | | | | | | X |
| DNA Wash | DNA wash solution I | X | X | X | X | X | X | X |
| | DNA wash solution II | X | X | X | X | X | X | X |
| DNA Elute | DNA elution solution | X | X | X | X | X | X | X |
| RNA Bind (750 μl) | Acetone/ethanol | X | X | X | X | X | X | X |
| RNA Wash | RNA wash solution | X | X | X | X | X | X | X |
| | Ethanol | X | X | X | X | X | X | X |
| RNA Elute | RNase-free water | X | X | X | X | X | X | X |
| Protein Bind (1400 μl) | Protein binding solution | X | X | X | X | X | X | X |
| Protein Wash | Protein wash solution | X | X | X | X | X | X | X |
| Protein Elute | Protein elution solution | X | X | X | X | X | X | X |

The yields and purity of isolated DNA and RNA are shown in the tables below.

### Qubits

| | | |
|---|---|---|
| Sample | Sample Concentration | Average |

| | | | |
|---|---|---|---|
| DNA | | | |
| A | 66.8 | ug/mL | |
| A | 66 | ug/mL | 66.4 |
| B | 58.1 | ug/mL | |
| B | 52.3 | ug/mL | 55.2 |
| C | 50.3 | ug/mL | |
| C | 52.3 | ug/mL | 51.3 |
| D | 40.5 | ug/mL | |
| D | 39.7 | ug/mL | 40.1 |
| E | 65.2 | ug/mL | |
| E | 53.7 | ug/mL | 59.5 |
| F | 61.1 | ug/mL | |
| F | 57.8 | ug/mL | 59.5 |
| G | 65.3 | ug/mL | |
| G | 64.3 | ug/mL | 64.8 |
| RNA | | | |
| A | 281 | ug/mL | |
| A | 341 | ug/mL | 311 |
| B | 315 | ug/mL | |
| B | 268 | ug/mL | 291.5 |
| C | 270 | ug/mL | |
| C | 236 | ug/mL | 253 |
| D | 261 | ug/mL | |
| D | 224 | ug/mL | 242.5 |
| E | 313 | ug/mL | |
| E | 299 | ug/mL | 306 |
| F | 248 | ug/mL | |
| F | 267 | ug/mL | 257.5 |
| G | 266 | ug/mL | |
| G | 323 | ug/mL | 294.5 |

### NanoDrop

| Sample | DNA(ng/uL) | A260/A280 | A260/A230 | A340 |
|---|---|---|---|---|
| A | 90.82 | 1.857 | 1.371 | −0.007 |
| A | 76.117 | 1.882 | 0.434 | 0.009 |
| B | 77.647 | 1.876 | 1.331 | 0.012 |
| B | 72.013 | 1.889 | 1.124 | 0.02 |
| C | 74.074 | 1.895 | 0.874 | 0.024 |
| C | 75.743 | 1.892 | 1.168 | 0.053 |
| D | 61.401 | 1.912 | 0.372 | 0.086 |
| D | 59.072 | 1.917 | 0.492 | 0.094 |
| E | 91.058 | 1.857 | 1.238 | 0.088 |
| E | 74.091 | 1.884 | 1.379 | 0.024 |
| F | 82.214 | 1.889 | 0.514 | 0.047 |
| F | 80.944 | 1.871 | 0.507 | 0.063 |
| G | 78.132 | 1.888 | 1.366 | 0.063 |
| G | 83.086 | 1.871 | 0.615 | 0.055 |

| Sample | RNA(ng/uL) | A260/A280 | A260/A230 | A340 |
|---|---|---|---|---|
| A | 468.983 | 2.019 | 0.867 | 0.475 |
| A | 469.489 | 2.02 | 0.874 | 0.669 |
| B | 492.453 | 2.033 | 0.851 | 0.515 |
| B | 421.741 | 2.025 | 0.89 | 0.339 |
| C | 387.829 | 2.013 | 0.696 | 0.292 |
| C | 326.908 | 2.008 | 0.771 | 0.28 |
| D | 330.422 | 2.015 | 0.877 | 0.259 |
| D | 309.391 | 1.999 | 0.739 | 0.277 |
| E | 531.691 | 2.029 | 0.855 | 0.601 |
| E | 608.165 | 2.008 | 1.105 | 0.574 |
| F | 404.415 | 2.023 | 0.765 | 0.309 |
| F | 444.787 | 2.022 | 0.91 | 0.462 |
| G | 369.487 | 2.017 | 0.895 | 0.316 |
| G | 530.529 | 2.018 | 0.896 | 0.419 |

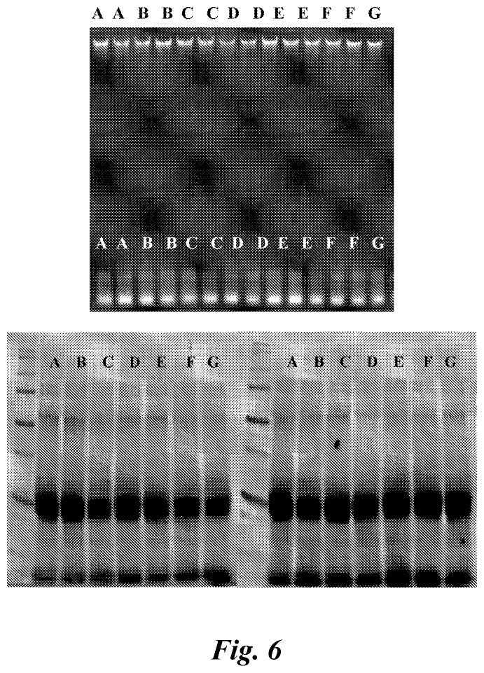

The gel electrophoresis of the isolated DNA, RNA and proteins is shown in FIG. 6, upper, middle and lower panels, respectively.

The results show that β-alanine was protective (i.e., reduced the reduction in yield) of DNA and RNA even at 0.12M AASD. The protein appeared unaffected by the addition of AASD with or without β-alanine.

### Example 7

### Exemplary Method for Isolating DNA, RNA and Proteins from Soil or Stool Samples

This example describes an exemplary method for isolating DNA, RNA and proteins from stool samples according to the present disclosure.

### Protocol in Summary

Up to 250 mg of stool are lysed via chemical and mechanical homogenization. Lysis buffer is added to a mixed zirconium bead tube containing the sample. Bead beating can be carried out using a standard benchtop vortex with bead tube adapter or the high-powered TissueLyzer. Crude lysate is then subjected to a single-step precipitation reaction to remove PCR and RT-PCR inhibitory compounds whilst retaining DNA, RNA and protein in solution. Following inhibitor removal, purified lysate is passed through a silica spin filter membrane to isolate total microbial DNA. A volume of RNA bind is added to the DNA flow-through and this solution is passed through a second spin column to capture total RNA. Finally, the RNA column flow-through is mixed with a low pH, high salt buffer to bind total proteins to a third spin column. All spin column membranes are washed and the DNA, RNA and proteins are eluted with dedicated elution reagents.

### Detailed Protocol

### Lysis:

1. Add up to 250 mg of stool into a ytrrium-stabilized zirconium mixed bead tube (0.1 and 0.5 mm beads, 1 gram of each).
2. Add 650 μl of lysis buffer (e.g., a buffer containing NaSCN and Na₂HPO₄) containing 6.5 μl beta-mercaptoethanol and 6.5 μl protease inhibitors (e.g., Halt protease inhibitors, ThermoFisher)
3. Vortex on high to mix.
4. Bead beat on maximum speed for 10 minutes.
5. Quick spin the tubes to drive fluid out of the bead tube cap.
6. Open the bead tubes and add 350 μl DNA binding solution I.
7. Cap the tubes and vortex on high to mix, approximately 1 minute.
8. Centrifuge bead tube for 1 minute @ 15,000×g.
9. Transfer supernatant (expect 800 μl) to a new collection tube.

### Inhibitor Removal:

10. Add 150 μl of inhibitor removal solution (e.g., a solution containing beta-alanine and AASD or a solution containing ammonium acetate and AASD).
11. Vortex on high to mix.
12. Centrifuge tube for 1 minute @ 15,000×g.
13. Transfer supernatant (expect 750 μl) to a new collection tube.

### DNA Binding:

14. Load 750 μl into a first spin column.
15. Centrifuge the first spin column for 1 minute @ 15,000×g. Retain flow-through, which contains RNA.
16. Place the spin column with immobilized DNA at +4° C. while RNA is being isolated.

### RNA Binding:

17. Add 750 μl of RNA binding solution I or RNA binding solution II to retained spin column flow-throughs from above and mix thoroughly.
18. Load 750 μl of RNA-containing lysate onto a second spin column.
19. Centrifuge the second spin column for 1 minute @ 15,000×g. Retain flow-through, which contains protein.
20. Repeat lysate loading and centrifugation until all lysate has been processed through the second spin column, retaining column flow-through each time.

### Protein Binding:

21. Combine all of the RNA spin column flow-through fractions into a single 5 mL conical tube.
22. Add 1400 μl of protein binding solution to the RNA flow-through volume and mix thoroughly.
23. Using a vacuum manifold, process the full volume of protein-containing lysate through a third spin column.

### Protein Washing:

24. With the third spin column still on the vacuum manifold, load 750 μl protein wash solution.

### Protein Elution:

25. Transfer the third spin column with bound proteins to new collection tubes.
26. Centrifuge the empty third spin column for 2 minutes @ 15,000×g.
27. Transfer the third spin column to new collection tube.
28. Elute bound proteins with 100 μl protein elution solution.

### DNA Washing:

29. Add 650 μl of DNA wash buffer I onto the first spin column.
30. Centrifuge the first spin column for 1 minute @ 15,000×g. Discard flow through.
31. Add 650 μl of DNA wash buffer II into the first spin column.
32. Centrifuge the first spin column for 1 minute @ 15,000×g. Discard flow through.
33. Centrifuge the empty first spin column for 2 minutes @ 15,000×g.
34. Transfer the first spin column to new collection tube.

### DNA Elution:

35. Add 100 μl of DNA elution solution to the center of the first spin column membrane.
36. Centrifuge the first spin column for 1 minute @ 15,000×g. Discard the first spin column.

### RNA Washing:

37. Add 650 μl of RNA wash solution onto the second spin column.
38. Centrifuge the second spin column for 1 minute @ 15,000×g. Discard flow through.
39. Load 650 μl of 100% ethanol onto the second spin column.
40. Centrifuge the second spin column for 1 minute @ 15,000×g. Discard flow through.
41. Centrifuge the empty second spin column for 2 minutes @ 15,000×g.
42. Transfer the second spin column to new collection tube.

### RNA Elution:

43. Add 100 μl of RNase-free water to the center of the second spin column membrane.
44. Centrifuge the second spin column for 1 minute @ 15,000×g. Discard the second spin column.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/662,066, filed Apr. 24, 2018, are incorporated herein by reference in their entirety except where incorporation of a reference or a portion thereof contradicts with the present disclosure. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for isolating proteins from a sample, comprising:
(a) contacting a sample, a lysate of the sample, a supernatant of the lysate, or a portion of the sample, the lysate or the supernatant with one or more first agents selected from low molecular weight carboxylates, low molecular weight sulfates, carboxylate polymers, sulfonated polymers, or mixtures thereof, and one or more second agents that are multivalent salt(s) to generate a mixture,
(b) separating the mixture of step (a) into a solid phase and a liquid phase, wherein the one or more first agents are primarily in the liquid phase, and wherein the one or more second agents are primarily in the solid phase, and
(c) isolating proteins from the liquid phase of step (b).

2. The method of claim 1, wherein the one or more first agents are selected from sulfoacetic acid, ammonium acetate, ammonium sulfate, ammonium glycolate, ammonium formate, beta-alanine, guanidine sulfate, histidine, glycine, sodium acetate, cesium acetate, and combinations thereof.

3. The method of claim 1, wherein the first agent is an amino acid or a low molecular weight carboxylate, such as sodium butyrate.

4. The method of claim 1, wherein the first agent is a carboxylate polymer, a sulfonated polymer, or a mixture thereof, such as sodium polystyrene sulfonate or sodium polyacrylic acid.

5. The method of any of claims 1 to 4, wherein the total concentration of the one or more first agents in the mixture of step (a) is in the range of 10 to 500 mM, such as 10 to 50 mM, 50 to 100 mM, 100 to 200 mM, 200 to 300 mM, 300 to 400 mM, 400 to 500 mM, 10 to 100 mM, 10 to 200 mM, 10 to 300 mM, 10 to 400 mM, 50 to 200 mM, 50 to 300 mM, 50 to 400 mM, 50 to 500 mM, 100 to 300 mM, 100 to 400 mM, 100 to 500 mM, 200 to 400 mM, 200 to 500 mM, or 300 to 500 mM, preferably 10 to 200 mM or 25 to 100 mM.

6. The method of any of claims 1 to 5, wherein the one or more second agents are selected from aluminum ammonium sulfate, aluminum ammonium sulfate dodecahydrate, aluminum chloride, aluminum sulfate, erbium (III) acetate, erbium (III) chloride, holmium chloride, zirconium (IV) chloride, hafnium (IV) chloride, and combinations thereof.

7. The method of any of claims 1 to 5, wherein the one or more second agents are selected from aluminum potassium sulfate, aluminum chlorohydrate, aluminum sulfate, calcium oxide, iron (III) chloride, iron (II) sulfate, magnesium chloride, and combinations thereof.

8. The method of any of claims 1 to 7, wherein the total concentration of the one or more second agents in the mixture of step (a) is in the range of 1 to 150 mM, such as 1 to 5 mM, 5 to 25 mM, 25 to 50 mM, 50 to 75 mM, 75 to 100 mM, 100 to 150 mM, 1 to 25 mM, 1 to 50 mM, 1 to 75 mM, 1 to 100 mM, 1 to 150 mM, 5 to 50 mM, 5 to 75 mM, 5 to 100 mM, 5 to 150 mM, 25 to 75 mM, 25 to 100 mM, 25 to 150 mM, 50 to 100 mM, 50 to 150 mM, 75 to 150 mM, preferably, 5 to 25 mM or 5 to 50 mM.

9. The method of any of claims 1 to 8, further comprising:
(d) isolating DNA, RNA, or both DNA and RNA from the liquid phase of step (b).

10. The method of claim 9, wherein steps (c) and (d) are performed sequentially.

11. The method of any one of claims 1 to 10, wherein the sample is a stool sample, a plant sample, or an environmental sample, such as a soil, water or air sample.

12. The method of any one of claims 1 to 11, wherein step (a) comprises contacting the sample, the lysate of the sample, the supernatant of the lysate, or the portion of the sample, the lysate or the supernatant with a composition that comprises the one or more first agents and the one or more second agents.

13. The method of any one of claims 1 to 11, wherein no precipitation, centrifugation, or filtration has been performed between contacting the sample, the lysate of the sample, or the supernatant of the lysate with the first agent and contacting the sample with the second agent.

14. The method of any one of claims 1 to 13, wherein step (a) is performed in the presence of a lytic reagent.

15. The method of claim 14, wherein the lytic reagent comprises one or more phosphates and one or more chaotropic agents selected from sodium thiocyanate, sodium carbonate, potassium thiocyanate, ammonium thiocyanate, lithium thiocyanate, lithium perchlorate, guanidine sulfate, and combinations thereof.

16. The method of claim 14, wherein the lytic reagent comprises sodium thiocyanate and sodium phosphate dibasic.

17. The method of any of claims 14 to 16, further comprising contacting a sample or a portion of a sample with the lytic reagent to generate a lysate of the sample, wherein step (a) comprises contacting the lysate of the sample, the supernatant of the lysate, or the portion of the lysate or the supernatant with the one or more first agents and the one or more second agents.

18. The method of any of claims 15 to 17, wherein the total concentration of the one or more chaotropic agents in the lytic reagent is in the range of 0.05 to 5M, 0.05 to 0.1M, 0.1 to 0.5M, 0.5 to 1M, 1 to 1.5M, 1.5 to 2M, 2 to 5 M, 0.1 to 1M, 0.1 to 1.5M, 0.1 to 2M, 0.1 to 5M, 0.5 to 1.5M, 0.5 to 2M, 0.5 to 5M, 1 to 2M, or 1 to 5M, preferably 0.05 to 0.5M or 0.5 to 2M.

19. The method of any of claims 15 to 18, wherein the total final concentration of the one or more chaotropic agents in the lysate is 0.01 to 4M, 0.01 to 0.05M, 0.05 to 0.1M, 0.1 to 0.5M, 0.5 to 1M, 1 to 1.5M, 1.5 to 2M, 2 to 4M, 0.01 to 0.1M, 0.01 to 0.5M, 0.01 to 1M, 0.01 to 1.5M, 0.01 to 2M, 0.01 to 4M, 0.05 to 0.5M, 0.05 to 1M, 0.05 to 1.5M, 0.05 to 2M, 0.05 to 2M, 0.05 to 4M, 0.1 to 1M, 0.1 to 1.5M, 0.1 to 2M, 0.1 to 4M, 0.5 to 1.5M, 0.5 to 2M, 0.5 to 4M, 1 to 2M, or 1 to 4M, preferably 0.05 to 0.5M or 0.5 to 2M.

20. The method of any of claims 15 to 19, wherein the total concentration of the one or more phosphates in the lytic reagent is 0.05 to 0.5M, preferably 0.1 to 0.2M.

21. The method of any of claims 15 to 20, wherein the total final concentration of the one or more phosphates in the lysate is 0.01 to 0.4M, preferably 0.1 to 0.2M.

22. The method of any one of claims 1 to 21, wherein the sample comprises an inhibitor, and the inhibitor is substantially precipitated and removed from the liquid phase of step (b) by the one or more second agents.

23. The method of any of claims 1 to 22, further comprising:
(e) analyzing the nucleic acids isolated in step (d).

24. The method of claim 23, wherein step (e) comprises performing PCR, qPCR, RT-PCR, or nucleic acid sequencing.

25. A method for sequentially separating and optionally isolating DNA, RNA and proteins from a sample, comprising:
(a) contacting a sample, a lysate of the sample, a supernatant of the lysate, or a portion of the sample, the lysate or the supernatant with one or more first agents selected from low molecular weight carboxylates, low molecular weight sulfates, carboxylate polymers, sulfonated polymers, or mixtures thereof, and one or more second agents that are multivalent salt(s) to generate a mixture;
(b) separating the mixture of step (a) into a solid phase and a liquid phase, wherein the one or more first agents are primarily in the liquid phase, and wherein the one or more second agents are primarily in the solid phase;
(c) separating and optionally isolating DNA from the liquid phase of step (b), comprising:
(1) contacting the liquid phase of step (b) with a first solid support under conditions so that DNA in the liquid phase of step (b) binds to the first solid support,
(2) optionally washing the DNA bound to the first solid support in step (c)(1), and
(3) optionally eluting the DNA optionally washed in step (c)(2) from the first solid support,
(d) separating and optionally isolating RNA from the flow through obtained from step (c)(1), comprising:
(1) contacting the flow through obtained from step (c)(1) with a second solid support under conditions so that RNA in the flow through obtained from step (c)(1) binds to the second solid support,
(2) optionally washing the RNA bound to the second solid support in step (d)(1), and
(3) optionally eluting the RNA optionally washed in step (d)(2) from the second solid support, and
(e) separating and optionally isolating protein from the flow through obtained from step (d)(1), comprising:
(1) contacting the flow through obtained from step (d)(1) with a third solid support under conditions so that proteins in the flow through obtained from step (d)(1) bind to the second solid support,
(2) optionally washing the proteins bound to the third solid support in step (e)(1), and
(3) optionally eluting the protein optionally washed in step (e)(2) from the third solid support.

26. The method of claim 25, further comprising contacting a sample or a portion of the sample with a lytic reagent to generate a lysate of the sample, wherein step (a) comprises contacting the lysate of the sample, the supernatant of the lysate, or the portion of the lysate or the supernatant with the one or more first agents and the one or more second agents.

27. The method of claim 26, wherein the lytic reagent comprises one or more phosphates and one or more chaotropic agents selected from sodium thiocyanate, sodium carbonate, potassium thiocyanate, ammonium thiocyanate, lithium thiocyanate, lithium perchlorate, guanidine sulfate, and combinations thereof.

28. The method of claim 27, wherein the lytic reagent comprises sodium thiocyanate and sodium phosphate dibasic.

29. The method of any of claims 25 to 28, wherein the one or more first agents are selected from amino acids; low molecular weight carboxylate, such as sodium butyrate; sodium polystyrene sulfonate; sodium polyacrylic acid; preferably sodium acetate, cesium acetate, sulfoacetic acid, ammonium acetate, ammonium sulfate, ammonium glycolate, ammonium formate, beta-alanine, guanidine sulfate, histidine, glycine, and combinations thereof.

30. The method of any of claims 25 to 29, wherein the one or more second agents are selected from aluminum ammonium sulfate, aluminum ammonium sulfate dodecahydrate, aluminum chloride, aluminum sulfate, erbium (III) acetate, erbium (III) chloride, holmium chloride, zirconium (IV) chloride, hafnium (IV) chloride, and combinations thereof.

31. The method of any of claims 25 to 29, wherein the one or more second agents are selected from aluminum potassium sulfate, aluminum chlorohydrate, aluminum sulfate, calcium oxide, iron (III) chloride, iron (II) sulfate, magnesium chloride, and combinations thereof.

32. The method of any of claims 25 to 31, wherein two or all of the first, second and third solid supports are identical to each other.

33. The method of any of claims 25 to 32, wherein the sample is a stool sample, a plant sample, or an environmental sample such as a soil, water, or air sample.

34. A composition for removing inhibitors during protein isolation from a sample, comprising, consisting essentially of, or consisting of:
(i) one or more first agents selected from low molecular weight carboxylates, low molecular weight sulfates, carboxylate polymers, sulfonated polymers, or mixtures thereof,
(ii) one or more second agents that are multivalent salt(s), and
(iii) optionally water,
wherein the one or more first agents are capable of maintaining water solubility upon coordination of the multivalent cation(s) of the one or more second agents, and
wherein
(A) the one or more first agents are selected from amino acids; salts of short chain fatty acids, such as sodium butyrate; sodium polystyrene sulfonate; sodium polyacrylic acid; preferably ammonium glycolate, sulfoacetic acid, ammonium formate, cesium acetate, beta-alanine, guanidine sulfate, histidine, glycine, and combinations thereof, and the one or more second agents are selected from aluminum sulfate, erbium (III) acetate, erbium (III) chloride, holmium chloride, zirconium (IV) chloride, hafnium (IV) chloride, aluminum potassium sulfate, aluminum chlorohydrate, calcium oxide, iron (III) chloride, iron (II) sulfate, magnesium chloride, aluminum ammonium sulfate, aluminum ammonium sulfate dodecahydrate, aluminum chloride, and combinations thereof,
OR
(B) the one or more first agents are selected from amino acids; salts of short chain fatty acids, such as sodium butyrate; sodium polystyrene sulfonate; sodium polyacrylic acid; preferably ammonium sulfate, ammonium glycolate, sulfoacetic acid, ammonium formate, sodium acetate, cesium acetate, ammonium acetate, beta-alanine, guanidine sulfate, histidine, glycine, and combinations thereof, and the one or more second agents are selected from erbium (III) acetate, erbium (III) chloride, holmium chloride, zirconium (IV) chloride, hafnium (IV) chloride, aluminum chloride, and combinations thereof.

35. The composition of claim 34, wherein the one or more first agents are selected from amino acids; salts of short chain fatty acids, such as sodium butyrate; sodium polystyrene sulfonate; sodium polyacrylic acid; preferably beta-alanine, guanidine sulfate, histidine, glycine, and combinations thereof, and the one or more second agents are selected from aluminum sulfate, erbium (III) acetate, erbium (III) chloride, holmium chloride, zirconium (IV) chloride, hafnium (IV) chloride, aluminum ammonium sulfate, aluminum ammonium sulfate dodecahydrate, aluminum potassium sulfate, aluminum chlorohydrate, calcium oxide, iron (III) chloride, iron (II) sulfate, magnesium chloride, aluminum chloride, and combinations thereof.

36. The composition of claim 34, wherein the one or more first agents are selected from amino acids; salts of short chain fatty acids, such as sodium butyrate; sodium polystyrene sulfonate; sodium polyacrylic acid; preferably ammonium sulfate, ammonium glycolate, sulfoacetic acid, ammonium formate, sodium acetate, cesium acetate, ammonium acetate, beta-alanine, guanidine sulfate, histidine, glycine, and combinations thereof, and the second agent is aluminum chloride.

37. The composition of any of claims 34 to 36, wherein the composition comprises water, the total concentration of the one or more first agents in the composition is 0.1 to 1M, and the total concentration of the one or more second agents in the composition is 10 to 500 mM.

38. The composition of claim 34 wherein the composition comprises, consists essentially of, or consists of:
(1) 0.2 to 0.8M guanidine sulfate and 20 to 200 mM aluminum ammonium sulfate or aluminum ammonium sulfate dodecahydrate;
(2) 0.25 to 1M beta-alanine and 20 to 200 mM aluminum ammonium sulfate or aluminum ammonium sulfate dodecahydrate;
(3) 0.25 to 1M glycine and 20 to 200 mM aluminum ammonium sulfate or aluminum ammonium sulfate dodecahydrate;
(4) 0.25 to 1M histidine and 20 to 200 mM aluminum ammonium sulfate or aluminum ammonium sulfate dodecahydrate;
(5) 0.2 to 0.8M guanidine sulfate and 20 to 200 mM aluminum chloride;
(6) 0.25 to 1M beta-alanine and 20 to 200 mM aluminum chloride;
(7) 0.25 to 1M glycine and 20 to 200 mM aluminum chloride; and
(8) 0.25 to 1M histidine and 20 to 200 mM aluminum chloride.

39. A kit for isolating proteins from a sample, comprising:
(a) the composition of any of claims 34 to 38,
OR
(b) (i) one or more first agents selected from low molecular weight carboxylates, low molecular weight sulfates, carboxylate polymers, sulfonated polymers, or mixtures thereof, and
(ii) one or more second agents that are multivalent salt(s),
wherein the one or more first agents are capable of maintaining water solubility upon coordination of the multivalent cation(s) of the one or more second agents, and
wherein
(A) the one or more first agents are selected from amino acids; salts of short chain fatty acids, such as sodium butyrate; sodium polystyrene sulfonate; sodium polyacrylic acid; preferably ammonium glycolate, sulfoacetic acid, ammonium formate, cesium acetate, beta-alanine, guanidine sulfate, histidine, glycine, and combinations thereof, and the one or more second agents are selected from aluminum sulfate, erbium (III) acetate, erbium (III) chloride, holmium chloride, zirconium (IV) chloride, hafnium (IV) chloride, aluminum potassium sulfate, aluminum chlorohydrate, calcium oxide, iron (III) chloride, iron (II) sulfate, magnesium chloride, aluminum ammonium sulfate, aluminum ammonium sulfate dodecahydrate, aluminum chloride, and combinations thereof,
OR
(B) the one or more first agents are selected from amino acids; salts of short chain fatty acids, such as sodium butyrate; sodium polystyrene sulfonate; sodium polyacrylic acid; preferably ammonium sulfate, ammonium glycolate, sulfoacetic acid, ammonium formate, sodium acetate, cesium acetate, ammonium acetate, beta-alanine, guanidine sulfate, histidine, glycine, and combinations thereof and the one or more second agents are selected from erbium (III) acetate, erbium (III) chloride, holmium chloride, zirconium (IV) chloride, hafnium (IV) chloride, aluminum chloride, and combinations thereof.

40. The kit of claim 39, wherein the kit comprises the composition of any of claims 34 to 38.

41. The kit of claim 39 or claim 40, further comprising a protein binding solid support.

42. The kit of any of claims 39 to 41, wherein the one or more first agents are selected from amino acids; salts of short chain fatty acids, such as sodium butyrate; sodium polystyrene sulfonate; sodium polyacrylic acid; preferably sodium acetate, cesium acetate, ammonium acetate, ammonium sulfate, ammonium glycolate, ammonium formate, beta-alanine, guanidine sulfate, histidine, glycine, and combinations thereof.

43. The kit of any of claims 39 to 41, wherein the one or more first agents are selected from beta-alanine, guanidine sulfate, histidine, glycine, and combinations thereof.

44. The kit of any of claims 39 to 43, wherein the one or more second agents are selected from aluminum sulfate, erbium (III) acetate, erbium (III) chloride, holmium chloride, zirconium (IV) chloride, hafnium (IV) chloride, aluminum potassium sulfate, aluminum chlorohydrate, calcium oxide, iron (III) chloride, iron (II) sulfate, magnesium chloride, aluminum ammonium sulfate, aluminum ammonium sulfate dodecahydrate, aluminum chloride, and combinations thereof.

45. The kit of any of claims 39 to 44, wherein the one or more second agents are selected from aluminum ammonium sulfate, aluminum ammonium sulfate dodecahydrate, aluminum chloride, aluminum sulfate, erbium (III) acetate, erbium (III) chloride, holmium chloride, zirconium (IV) chloride, hafnium (IV) chloride, and combinations thereof.

46. The kit of any of claims 39 to 45, wherein the first agent is beta-alanine or guanidine sulfate, and the second agent is selected from aluminum ammonium sulfate, aluminum ammonium sulfate dodecahydrate, aluminum chloride, and combinations thereof.

47. The kit of any of claims 39 to 46, wherein the protein-binding solid support is a protein-binding spin column.

48. The kit of any of claims 39 to 47, further comprising one or more of a protein binding solution, a protein wash solution, and a protein elution solution.

49. The kit of any of claims 39 to 48, further comprising a lytic reagent.

50. The kit of claim 49, wherein the lytic reagent comprises one or more phosphates and one or more chaotropic agents.

51. The kit of claim 50, wherein the total concentration of the one or more chaotropic agents in the lytic reagent is in the range of 0.05 to 5M, 0.05 to 0.1M, 0.1 to 0.5M, 0.5 to 1M, 1 to 1.5M, 1.5 to 2M, 2 to 5 M, 0.1 to 1M, 0.1 to 1.5M, 0.1 to 2M, 0.1 to 5M, 0.5 to 1.5M, 0.5 to 2M, 0.5 to 5M, 1 to 2M, or 1 to 5M, preferably 0.05 to 0.5M or 0.5 to 2M, and the total concentration of the one or more phosphates is 0.05 to 0.5M, preferably 0.1 to 0.2M.

52. The kit of claim 50 or claim 51, wherein the one or more chaotropic agents are selected from sodium thiocyanate, sodium carbonate, potassium thiocyanate, ammonium thiocyanate, lithium thiocyanate, lithium perchlorate, guanidine sulfate, and combinations thereof.

53. The kit of claim 49, wherein the lytic reagent comprises sodium phosphate dibasic and sodium thiocyanate.

54. The kit of any of claims 39 to 53, further comprising a nucleic acid-binding solid support.

55. The kit of any of claims 39 to 54, further comprising one or more of the solutions selected from DNA binding solution, DNA wash solution, DNA elution solution, RNA binding solution, RNA wash solution, and RNA elution solution.
